(19) United States
(12) Patent Application Publication
(10) Pub. No.: US 2025/0302800 A1
(43) Pub. Date: Oct. 2, 2025

(54) METHODS OF TREATING SOCIAL FUNCTION DISORDERS

(71) Applicant: Sumitomo Pharma America, Inc., Marlborough, MA (US)

(72) Inventor: David Blum, Marlborough, MA (US)

(73) Assignee: Sumitomo Pharma America, Inc., Marlborough, MA (US)

(21) Appl. No.: 18/987,706

(22) Filed: Dec. 19, 2024

Related U.S. Application Data
(63) Continuation of application No. 18/738,187, filed on Jun. 10, 2024, now abandoned.
Application No. 18/738,187 is a continuation of application No. 18/192,234, filed on Mar. 29, 2023, now abandoned.
Application No. 18/192,234 is a continuation of application No. 17/931,587, filed on Sep. 13, 2022, now abandoned.
Application No. 17/931,587 is a continuation of application No. 16/970,089, filed on Aug. 14, 2020, now abandoned.
(60) Provisional application No. 62/761,253, filed on Feb. 16, 2018.

(51) Int. Cl. A61K 31/381; A61K 47/10; A61K 47/14; A61K 47/38

(52) U.S. Cl. CPC A61K 31/381; A61K 47/10; A61K 47/14; A61K 47/38

(57) ABSTRACT

Methods and compositions for treating social function disorders are disclosed. The methods involve administering compound of Formula I

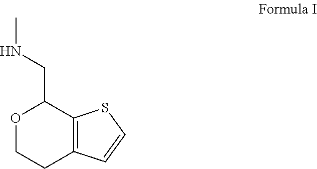

### CROSS-REFERENCE TO RELATED APPLICATIONS

This application claims priority from U.S. provisional application 62/761,253, filed Feb. 16, 2018, which is incorporated herein by reference in its entirety.

### FIELD

The present application relates generally to methods and compositions for treating social function disorders comprising administering compounds disclosed herein.

### BACKGROUND

Social function disorders, such as neurodevelopmental disorders, obsessive-compulsive disorders and disruptive, impulse-control and conduct disorders can impair how an individual functions socially. See, e.g. Diagnostic and Statistical Manual of Mental Disorders, Fifth Edition (DSM-5). Development of a therapeutically effective pharmaceutical compound may help reduce, eliminate or prevent social function disorders or symptoms thereof. Accordingly, a therapeutically effective and chemically stable pharmaceutical compound that treats or prevents a social function disorder, such as a neurodevelopmental disorder, an obsessive-compulsive disorder or a disruptive, impulse-control and conduct disorder is desired.

### SUMMARY

Provided herein is a method of treating or preventing a social function disorder comprising administering to a subject in need thereof a therapeutically effective amount of

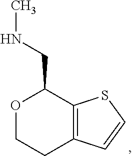

or a pharmaceutically acceptable salt thereof.

In some embodiments, the social function disorder is a neurodevelopmental disorder, an obsessive-compulsive disorder or a disruptive, impulse-control and conduct disorder.

In some embodiments, the social function disorder is a language disorder, a speech sound disorder, a childhood-onset fluency disorder (stuttering), a social communication disorder, a developmental coordination disorder, a stereotypical movement disorder, a tic disorder, Tourette's disorder, a persistent (chronic) motor or vocal tic disorder, a provisional tic disorder, an other specified tic disorder, an unspecified tic disorder, an obsessive-compulsive disorder, or an impulse-control disorder.

In some embodiments, the social function disorder is a language disorder, a speech sound disorder, a childhood-onset fluency disorder (stuttering), a social communication disorder, a developmental coordination disorder, a stereotypical movement disorder, a tic disorder, Tourette's disorder, a persistent (chronic) motor or vocal tic disorder, a provisional tic disorder, an other specified tic disorder, or an unspecified tic disorder.

In some embodiments, the social function disorder is a language disorder, a speech sound disorder, a childhood-onset fluency disorder (stuttering), or a social communication disorder.

In some embodiments, the social function disorder is a language disorder, childhood-onset fluency disorder (stuttering), social communication disorder, developmental coordination disorder, stereotypical movement disorder, persistent (chronic) motor or vocal tic disorder, provisional tic disorder, other specified tic disorder, or unspecified tic disorder.

In some embodiments, provided is a method of treating or preventing a social function disorder comprising administering to a subject in need thereof a pharmaceutical composition comprising:
(a) 25 mg to 125 mg of a compound of the following formula:

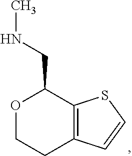

or a pharmaceutically acceptable salt thereof;
(b) one or more filler;
(c) one or more disintegrant; and
(d) one or more lubricant.

In some embodiments, the one or more filler is any one or more of microcrystalline cellulose, mannitol, and xylitol.

In some embodiments, the one or more disintegrant is sodium starch glycolate.

In some embodiments, the one or more lubricant is magnesium stearate.

In some embodiments, provided is a method of treating or preventing a neurodevelopmental disorder, an obsessive-compulsive disorder or a disruptive, impulse-control and conduct disorder comprising administering to a subject in need thereof a pharmaceutical composition comprising:
(a) 30 to 125 mg of (S)-Formula I HCl Form A;
(b) 100 to 250 mg of Microcrystalline Cellulose;
(c) 25 to 100 mg of Mannitol;
(d) 5 to 10 mg of Sodium Starch Glycolate; and
(e) 0.75 to 2 mg of Magnesium Stearate.

### BRIEF DESCRIPTION OF THE DRAWINGS

In the accompanying drawings (e.g., FIG. 1A, FIG. 1B, etc.), matching reference numerals indicate like elements and features in the various drawings. For clarity, not every element may be labeled in every drawing. In addition, the drawings are not necessarily complete when viewed without reference to the text.

The following abbreviations are used herein. The abbreviation DSC refers to differential scanning calorimetry; the abbreviation XRD refers to x-ray diffraction; the abbreviation XRPD refers to x-ray powder diffraction; the abbreviation NMR refers to nuclear magnetic resonance; the abbreviation DVS refers to dynamic vapor sorption; the abbreviation FBRM refers to focused beam reflectance measurement; the abbreviation HPLC refers to high performance liquid chromatography; and the abbreviation GC refers to gas chromatography; the abbreviation PSD refers to particle size distribution; the abbreviations D4,3 and D(4,3) refer to the volume mean diameter of a volume percent PSD; the abbreviation D50 refers to the median of a distribution where half the population resides above this value and half resides below; the abbreviation D10 refers to the point on a distribution where 10% of the population resides below this value; the abbreviation D90 refers to the point on a distribution where 90% of the population resides below this value; the abbreviation PVM refers to particle vision and measurement. Other abbreviations not explicitly described herein have their normal meanings in the art.

FIG. 1A, FIG. 1B, FIG. 1C, and FIG. 1D present SEM images of crystalline (S)-1-(4,7-dihydro-5H-thieno[2,3-c]pyran-7-yl)-N-methylmethanamine hydrochloride (“(S)-Formula I HCl”): Crystalline (S)-Formula I HCl Form A (FIG. 1A and FIG. 1B) and Crystalline (S)-Formula I HCl Form B (FIG. 1C and FIG. 1D).

FIG. 2A and FIG. 2B present XRPD patterns for (S)-Formula I HCl Form A; FIG. 2A is the XRPD measured in transmission mode and FIG. 2B in reflection mode.

FIG. 2C presents an XRPD pattern measured in reflection mode for (S)-Formula I HCl Form B.

FIG. 3A is a DSC thermogram for (S)-Formula I HCl Form A.

FIG. 3B and FIG. 3C are DSC thermograms for (S)-Formula I HCl Form B.

FIG. 4A, FIG. 4B, FIG. 4C, FIG. 4D, and FIG. 4E present various types of Raman spectra of for (S)-Formula I HCl Forms A and B; where FIG. 4A presents Raman spectra of Form A; where FIG. 4B presents Raman spectra of Form B; where FIG. 4C presents Raman spectra of both Form A (lower trace) and Form B (upper trace); FIG. 4D presents a Terahertz (THz) Raman spectra of Form A peak at 1089 cm⁻¹ (wavenumbers); and FIG. 4E presents a Terahertz (THz) Raman spectra of Form B peak at 1162 cm⁻¹ (wavenumbers).

FIG. 5 is a DVS water sorption isotherm for (S)-Formula I HCl Form A.

FIG. 6A and FIG. 6B present various HCl dosing profiles data of Example 2 for (S)-Formula I HCl Form A.

FIG. 7A and FIG. 7B present various PSD (particle size distribution) data of Example 2 for (S)-Formula I HCl Form A.

FIG. 8A presents PSD (particle size distribution) data of Example 2 for (S)-Formula I HCl Form A at 15.2% free base.

FIG. 8B presents PSD data of Example 2 for (S)-Formula I HCl Form A at 13% free base.

FIG. 8C presents PSD data of Example 2 for (S)-Formula I HCl Form A at 10.8% free base.

FIG. 9A presents various PSD (particle size distribution) data of Example 2 for (S)-Formula I HCl Form A.

FIG. 9B and FIG. 9C present SEM images of crystalline (S)-Formula I HCl Form A.

FIG. 10 is a ¹H NMR spectrum of (S)-Formula I HCl Form A.

### DETAILED DESCRIPTION

The description herein is made with the understanding that the present disclosure is to be considered as an exemplification of the claimed subject matter, and is not intended to limit the appended claims to the specific embodiments illustrated. The headings used throughout this disclosure are provided for convenience and are not to be construed to limit the claims in any way. Embodiments illustrated under any heading may be combined with embodiments illustrated under any other heading.

All published documents cited herein are hereby incorporated by reference in their entirety.

### Definitions

Unless defined otherwise, all technical and scientific terms used herein have the same meaning as commonly understood by one of ordinary skill in the art.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”. The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition or method.

As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has” and “having”), “include” (and any form of include, such as “includes” and “including”), and “contain” (and any form contain, such as “contains” and “containing”) are open-ended linking verbs. As a result, a method that “comprises”, “has”, “includes” or “contains” one or more steps or elements possesses those one or more steps or elements, but is not limited to possessing only those one or more steps or elements.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another.

A “tautomer” refers to a proton shift from one atom of a molecule to another atom of the same molecule. The present disclosure includes tautomers of any said compounds.

A “solvate” is formed by the interaction of a solvent and a compound. Solvates of salts of the compounds described herein are also provided. Hydrates of the compounds described herein are also provided.

A “prodrug” includes any compound that becomes a compound described herein when administered to a subject, e.g., upon metabolic processing of the prodrug.

As used herein, the term “subject,” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. The “subject” may have independently been diagnosed with a disorder as defined herein, may currently be experiencing symptoms associated with disorders or may have experienced symptoms in the past, may be at risk of developing a disorder, or may be reporting one or more of the symptoms of a disorder, even though a diagnosis may not have been made.

As used herein, the term “therapeutically effective amount” or “effective amount” refers to an amount that is effective to elicit the desired biological or medical response, including the amount of a compound that, when administered to a subject for treating a disorder, is sufficient to effect such treatment of the disorder. The effective amount will vary depending on the compound, the disorder, and its severity, and the age, weight, etc. of the subject to be treated. The effective amount may be in one or more doses (for example, a single dose or multiple doses may be required to achieve the desired treatment endpoint). An effective amount may be considered to be given in an effective amount if, in conjunction with one or more other agents, a desirable or beneficial result may be or is achieved. Suitable doses of any co-administered compounds may optionally be lowered due to the combined action, additive or synergistic, of the compound.

“Pharmaceutically acceptable” or “physiologically acceptable” refer to compounds, salts, compositions, dosage forms and other materials which are useful in preparing a pharmaceutical composition that is suitable for veterinary or human pharmaceutical use.

As used herein, the term “pharmaceutically acceptable excipient” includes, without limitation, any binder, filler, adjuvant, carrier, excipient, glidant, sweetening agent, diluent, preservative, dye/colorant, flavor enhancer, surfactant, wetting agent, dispersing agent, suspending agent, stabilizer, isotonic agent, solvent, emulsifier, anti-caking agent, flavor, desiccant, plasticizer, vehicle, disintegrant, or lubricant which has been approved by the United States Food and Drug Administration as being acceptable for use in humans or domestic animals.

In certain embodiments, non-limiting examples of excipients include corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, talc, calcium carbonate (e.g., granules or powder), sodium carbonate, microcrystalline cellulose, powdered cellulose, dextrates, kaolin, mannitol, silicic acid, sorbitol, starch, pre-gelatinized starch, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, a syloid silica gel (AEROSIL200, manufactured by W.R. Grace Co. of Baltimore, MD), a coagulated aerosol of synthetic silica (marketed by Degussa Co. of Plano, TX), CAB-O-SIL (a pyrogenic silicon dioxide product sold by Cabot Co. of Boston, MA), and mixtures thereof.

As used herein, the terms “treatment” or “treating” are used interchangeably. These terms refer to an approach for obtaining beneficial or desired results including, but not limited to, therapeutic benefit. Therapeutic benefit includes eradication and/or amelioration of the underlying disorder being treated; it also includes the eradication and/or amelioration of one or more of the symptoms associated with the underlying disorder such that an improvement is observed in the subject, notwithstanding that the subject may still be afflicted with the underlying disorder. In some embodiments, “treatment” or “treating” includes one or more of the following: (a) inhibiting the disorder (for example, decreasing one or more symptoms resulting from the disorder, and/or diminishing the extent of the disorder); (b) slowing or arresting the development of one or more symptoms associated with the disorder (for example, stabilizing the disorder and/or delaying the worsening or progression of the disorder); and/or (c) relieving the disorder (for example, causing the regression of clinical symptoms, ameliorating the disorder, delaying the progression of the disorder, and/or increasing quality of life.)

As used herein, the term “disorder” or specifically identified disorders disclosed herein, (e.g. neurodevelopmental disorder, obsessive-compulsive disorder, disruptive, impulse-control and conduct disorder) refer to the disorder as defined in the Diagnostic and Statistical Manual of Mental Disorders, Fifth Edition (DSM-5).

As used herein, the term “social function disorder” refers to any disorder defined in the Diagnostic and Statistical Manual of Mental Disorders, Fifth Edition (DSM-5) that may affect a subject's function socially (e.g., a social function disorder may impair a subject's ability to communicate with others by, for example, hindering speech, triggering impulses, or limiting self-control). In some embodiments, the term social function disorder refers to a “neurodevelopmental disorder”, an “obsessive-compulsive disorder” or a “disruptive, impulse-control and conduct disorder” as defined in Section II of the Diagnostic and Statistical Manual of Mental Disorders, Fifth Edition (DSM-5). The DSM-5 defines neurodevelopmental disorders as a group of conditions with onset in the developmental period, typically manifesting in early in development, often before a child enters grade school, and are characterized by developmental deficits that produce impairments of personal, social, academic, or occupational functioning. As used herein, “social function disorder” encompasses neurodevelopmental disorders. The DSM-5 defines obsessive-compulsive disorders as obsessive-compulsive disorder (OCD), body dysmorphic disorder, hoarding disorder, trichotillomania (hair-pulling disorder), excoriation (skin-picking) disorder, substance/medication-induced obsessive-compulsive and related disorder, obsessive-compulsive and related disorder due to another medical condition, and other specified obsessive-compulsive and related disorder and unspecified obsessive-compulsive and related disorder (e.g., body-focused repetitive behavior disorder, obsessional jealousy). As used herein, “social function disorder” encompasses obsessive-compulsive disorders. The DSM-5 defines disruptive, impulse-control, and conduct disorders as problems that are manifested in behaviors that violate the rights of others (e.g., aggression, destruction of property) and/or that bring the individual into significant conflict with societal norms or authority figures. As used herein, “social function disorder” encompasses disruptive, impulse-control, and conduct disorders.

As used herein, “delaying” development of a disorder mean to defer, hinder, slow, stabilize, and/or postpone development of the disorder. Delay can be of varying lengths of time, depending on the history of the disease and/or the individual being treated.

As used herein, “prevention” or “preventing” refers to a regimen that protects against the onset of the disorder such that the clinical symptoms of the disorder develop to a lesser extent than they would in the absence of treatment. Accordingly, “prevention” relates to administration of a therapy, including administration of a compound disclosed herein, to a subject before signs of the diseases are detectable in the subject (for example, administration of a compound disclosed herein to a subject in the absence of a detectable syndrome of the disorder). The subject may be an individual at risk of developing the disorder.

As used herein, an “at risk” individual is an individual who is at risk of developing a disorder to be treated. This may be shown, for example, by one or more risk factors, which are measurable parameters that correlate with development of a disorder and are known in the art.

As used herein, the term “polymorph” refers to different crystal structures achieved by a particular chemical entity. As used herein, the term “solvate” refers to a crystal form where a stoichiometric or non-stoichiometric amount of solvent, or mixture of solvents, is incorporated into the crystal structure. Similarly, the term “hydrate” refers to a crystal form where a stoichiometric or non-stoichiometric amount of water is incorporated into the crystal structure.

Polymorphism is the ability of an element or compound to crystallize into distinct crystalline phases. Although the term polymorph implies more than one morphology, the term is still used in the art, and herein, to refer to a crystalline structure of a compound as a polymorph even when only one crystalline phase is currently known. Thus, polymorphs are distinct solids sharing the same molecular formula as other polymorphs and the amorphous (non-crystalline) phase, however since the properties of any solid depend on its structure, polymorphs often exhibit physical properties distinct from each other and the amorphous phase, such as different solubility profiles, different melting points, different dissolution profiles, different thermal stability, different photostability, different hygroscopic properties, different shelf life, different suspension properties and different physiological absorption rates. Inclusion of a solvent in the crystalline solid leads to solvates, and in the case of water as a solvent, hydrates, often leads to a distinct crystalline form with one or more physical properties that are distinctly different from the non-solvated and non-hydrated (e.g., anhydrous) crystalline form.

As used herein the term “span,” when referring to a PSD is evaluated as follows: Span=[(D90−D10)/D50], for D values of a PSD distribution based on volume.

As used herein, the term “prominent peak,” in the context of an XRPD, means a peak with a greater than about 15% relative intensity. As used herein, the term “insignificant peak,” in the context of an XRPD, means a peak with a less than about 2% relative intensity.

As used herein the term “polymorph purity” refers to the weight % that is the specified polymorph form. For example, when a crystalline compound (e.g. Form A) is characterized as having greater than 95% polymorph purity, that means that greater than 95% by weight of the substance is the crystalline compound Form A and less than 5% by weight of any other polymorph (e.g., Form B) or amorphous form of the crystalline compound.

As used herein the terms “chiral purity” and “enantiomeric purity” are used interchangeably and refers to the weight % that is the specified enantiomer. For example, when an enantiomer-containing substance (such as a compound or crystal) is characterized as having greater than 90% chiral purity, that means that greater than 95% by weight of the substance is the specific enantiomer and less than 5% by weight is in any other enantiomeric form.

As used herein the term “chemical purity” refers to the weight % that is the specified chemical entity, including specified enantiomeric or polymorph form. For example, when a crystalline form (e.g. Form A) is characterized as having greater than 95% chemical purity, that means that greater than 95% by weight of the substance is the crystalline form (e.g. Form A) and less than 5% by weight of any other compound including other enantiomers and polymorphs.

As used herein “chemically stable” in reference to a pharmaceutical composition, describes a pharmaceutical composition that is resistant to decomposition when exposed to natural conditions, such as air, heat, light, pressure, or humidity for a period of time. In some embodiments, the period of time may be more than one week or more than two weeks or more than three weeks or more than four weeks or more than one month or more than two months or more than three months or more than four months or more than five months or more than six months. In some non-limiting examples, a chemically stable pharmaceutical composition is resistant to decomposition when exposed to air, heat, light, pressure, or humidity for more than one week or more than two weeks or more than three weeks or more than four weeks or more than one month or more than two months or more than three months or more than four months or more than five months or more than six months.

### Compounds

The present disclosure provides a compound of Formula I:

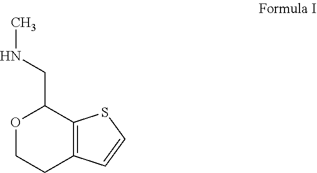

One of ordinary skill in the art would appreciate that nomenclature of compounds may vary. The compound of Formula I has an IUPAC name 1-(4,7-dihydro-5H-thieno[2,3-c]pyran-7-yl)-N-methylmethanamine. The compound of Formula I has a CAS registry number 1310426-29-9.

Preparation of the compound of Formula I can be found in PCT Publication No. WO2011069063, for example, on page 143, example 89, which is incorporated herein in its entirety.

The “compound of Formula I” includes stereoisomers (e.g. a compound of Formula I includes, but is not limited to, a racemate and each stereoisomer).

In some embodiments, the compound of Formula I is the stereoisomer (S)-1-(4,7-dihydro-5H-thieno[2,3-c]pyran-7-yl)-N-methylmethanamine (“(S)-Formula I”);

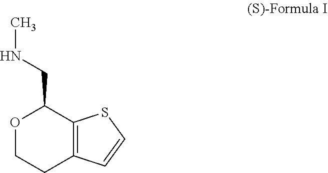

Preparation of the compound (S)-Formula I can be found in PCT Publication No. WO2011069063, for example, on page 151, example 129, which is incorporated herein in its entirety.

In some embodiments, the compound of Formula I is the stereoisomer (R)-1-(4,7-dihydro-5H-thieno[2,3-c]pyran-7-yl)-N-methylmethanamine (“(R)-Formula I”):

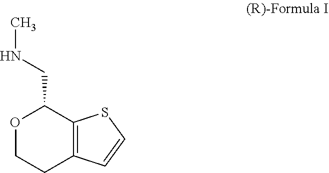

Preparation of the compound (R)-Formula I can be found in PCT Publication No. WO2011069063, for example, on page 151, example 128, which is incorporated herein in its entirety.

Amounts of the compound of Formula I described herein, unless otherwise defined, are the amount calculated as the free base. The amounts can be adjusted according to the salt form of being employed. For example, 118.6 mg of a hydrochloride salt of the compound of Formula I can be equivalent to 100 mg of the free base.

Provided are also pharmaceutically acceptable salts, hydrates, solvates, tautomeric forms, polymorphs, and prodrugs of the compounds described herein.

The compounds described herein may be prepared and/or formulated as pharmaceutically acceptable salts.

For example, a pharmaceutically acceptable salt of a compound of Formula I would include

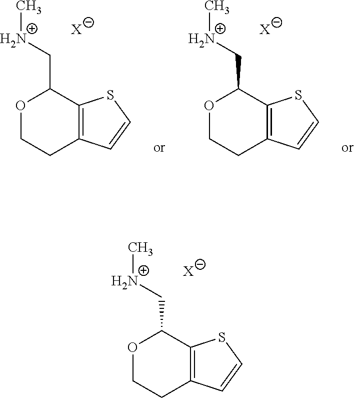

in which X⁻ is any counterion. In certain embodiments, X⁻ is the conjugate base of a pharmaceutically acceptable acid.

Pharmaceutically acceptable salts are non-toxic salts of a free base form of a compound that possesses the desired pharmacological activity of the free base. These salts may be derived from inorganic or organic acids or bases. For example, a compound that contains a basic nitrogen may be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, .gamma.-hydroxybutyrates, glycolates, tartrates, and mandelates. Lists of other suitable pharmaceutically acceptable salts are found in Remington: The Science and Practice of Pharmacy, 21.sup.st Edition, Lippincott Williams and Wilkins, Philadelphia, Pa., 2006.

Examples of “pharmaceutically acceptable salts” of the compounds disclosed herein also include salts derived from an appropriate base, such as an alkali metal (for example, sodium, potassium), an alkaline earth metal (for example, magnesium), ammonium and NX₄⁺ (wherein X is C₁-C₄ alkyl). Also included are base addition salts, such as sodium or potassium salts.

Provided are also compounds described herein or pharmaceutically acceptable salts, isomers, or a mixture thereof, in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds may increase resistance to metabolism, and thus may be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism”, Trends Pharmacol. Sci., 5(12):524-527 (1984). Such compounds are synthesized by means well known in the art, for example by employing starting materials in which one or more hydrogen atoms have been replaced by deuterium.

Examples of isotopes that can be incorporated into the disclosed compounds also include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula I, can generally be prepared by conventional techniques known to those skilled in the art or by processes analogous to those described in the Examples as set out below using an appropriate isotopically-labeled reagent in place of the non-labeled reagent previously employed.

The compounds disclosed herein, or their pharmaceutically acceptable salts, may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Likewise, all tautomeric forms are also intended to be included.

### Crystalline Forms

The compounds disclosed herein, or their pharmaceutically acceptable salts, may exist in crystalline forms. As disclosed herein, the compound (S)-Formula I HCl is polymorphic and exists in two crystalline forms, (S)-Formula I HCl Form A and (S)-Formula I HCl Form B. Form A was found to be thermodynamically stable, not substantially converting to other polymorphs or an amorphous form. Formation of Form B was found to be kinetically favored over Form A, however, was also found to be less thermodynamically stable than Form A; Form B being transformed to Form A when Form B is held as a slurry and slightly heated.

Crystalline forms of (S)-Formula I and (S)-Formula I HCl and crystalline forms of other salts, hydrates and solvates, including those of the present disclosure, may be characterized and differentiated using a number of conventional analytical techniques, including but not limited to X-ray powder diffraction (XRPD) patterns, nuclear magnetic resonance (NMR) spectra, Raman spectra, Infrared (IR) absorption spectra, dynamic vapor sorption (DVS), Differential Scanning calorimetry (DSC), and melting point. Chemical purity may be characterized using a number of conventional analytical techniques, including but not limited to high performance liquid chromatography (HPLC) and gas chromatography (GC). Chiral purity (also known as enantiomeric purity) may be characterized using a number of conventional analytical techniques, including but not limited to high performance liquid chromatography (HPLC).

In some embodiments, the crystalline forms of (S)-Formula I HCl are characterized by X-ray powder diffraction (XRPD). XRPD is a technique of characterizing a powdered sample of a material by measuring the diffraction of X-rays by the material. The result of an XRPD experiment is a diffraction pattern. Each crystalline solid produces a distinctive diffraction pattern containing sharp peaks as a function of the scattering angle 2-θ (2-theta). Both the positions (corresponding to lattice spacing) and the relative intensity of the peaks in a diffraction pattern are indicative of a particular phase and material. This provides a “fingerprint” for comparison to other materials. In contrast to a crystalline pattern comprising a series of sharp peaks, amorphous materials (liquids, glasses etc.) produce a broad background signal in a diffraction pattern.

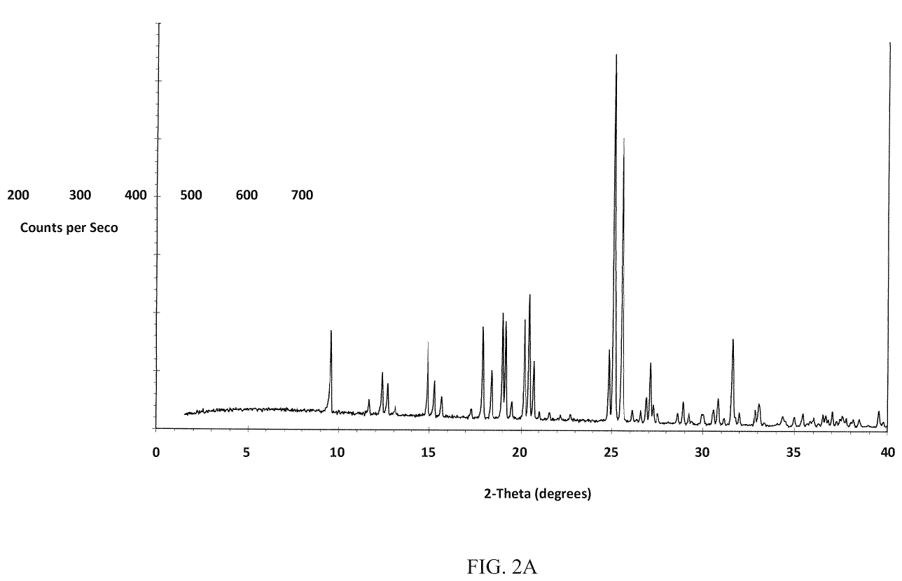

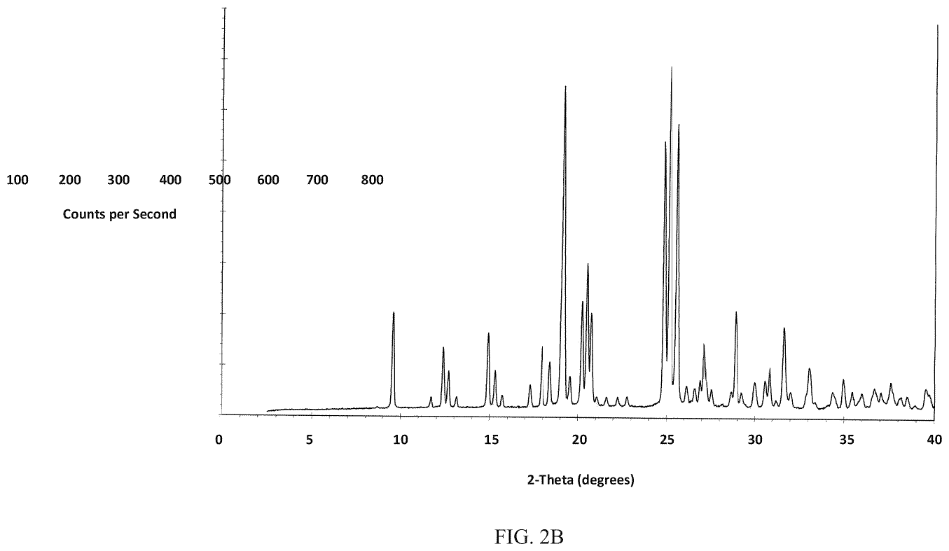

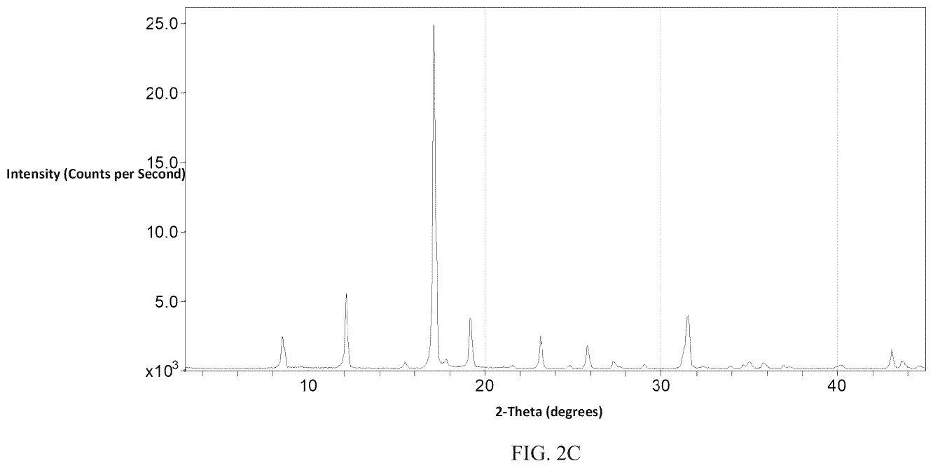

One of ordinary skill in the art would understand that certain parameters (e.g. the apparatus employed, humidity, temperature, orientation of the powder crystals, etc.) involved in obtaining an XRPD pattern may cause some variability in the appearance, intensities, and positions of the lines in the diffraction pattern. An XRPD pattern that is “substantially in accord with” that of a Figure provided herein (e.g., FIG. 2A) is an XRPD pattern that would be considered by one skilled in the art to represent a compound possessing the same crystal form as the compound that provided the XRPD pattern of that Figure. That is, the XRPD pattern may be identical to that of the Figure, or more likely it may be somewhat different. Such an XRPD pattern may not necessarily show each of the lines of the diffraction patterns presented herein, and/or may show a slight change in appearance, intensity, or a shift in position of said lines resulting from differences in the conditions involved in obtaining the data. A person skilled in the art is capable of determining if a sample of a crystalline compound has the same form as, or a different form from, a form disclosed herein by comparison of their XRPD patterns.

For example, one skilled in the art could use HPLC to determine the enantiomeric identity of a sample comprising a compound of Formula I HCl and if, for example, the sample is identified as (S)-Formula I HCl, one skilled in the art can overlay an XRPD pattern of the sample with FIG. 2A and/or FIG. 2B, and using expertise and knowledge in the art, readily determine whether the XRPD pattern of the sample is substantially in accordance with the XRPD pattern of crystalline (S)-Formula I HCl Form A as presented in FIG. 2A or (S)-Formula I HCl Form B as presented in FIG. 2B, or neither. If, for example, HPLC identifies the sample as being (S)-Formula I HCl and the sample XRPD pattern is substantially in accord with FIG. 2A, the sample can readily and accurately be identified as (S)-Formula I HCl Form A.

In various embodiments, the crystalline forms of (S)-Formula I HCl are characterized by Raman Spectroscopy and THz Raman Spectroscopy. The positions and the relative intensity of the peaks are indicative of the vibrational, and other low frequency modes, of a compound and can provides a “fingerprint” for comparison to other compounds. THz Raman spectroscopy provides further “fingerprint” information by extending the range into the terahertz frequency region of both Stokes and anti-Stokes signals, and THz Raman spectroscopy in general providing greater structural information, such as distinguishing between polymorphs, than Raman spectroscopy.

In some embodiments, the crystalline forms of (S)-Formula I HCl are characterized by melting point. Melting points were determined by conventional methods such as capillary tube and may exhibit a range over which complete melting occurs, or in the case of a single number, a melt point of that temperature ±1° C.

In some embodiments, the crystalline forms of (S)-Formula I HCl are characterized by differential scanning calorimetry (DSC). DSC is a thermoanalytical technique in which the difference in the amount of heat required to increase the temperature of a sample and a reference is measured as a function of temperature. Both the sample and reference are maintained at substantially the same temperature throughout the experiment. The result of a DSC experiment is a curve of heat flow versus temperature, called a DSC thermogram.

In some embodiments, the hygroscopicity of crystal forms of (S)-Formula I HCl are characterized by dynamic vapor sorption (DVS). DVS is a gravimetric technique that measures how much of a solvent is absorbed by a sample by varying the vapor concentration surrounding the sample (e.g., relative humidity) and measuring the change in mass. In the present application, DVS is used to generate water sorption isotherms, which represent the equilibrium amount of vapor sorbed as a function of steady state relative vapor pressure at a constant temperature.

As used herein, the term “substantially non-hygroscopic” refers to a compound exhibiting less than a 1% maximum mass change in water sorption isotherms, at 25° C. scanned over 0 to 90%/o relative humidity, as measured by dynamic vapor sorption (DVS).

In some embodiments, the present disclosure relates to new crystalline forms of (S)-Formula I HCl (e.g. Form A and Form B). Form A has been found to be a distinct polymorph from Form B, having a distinctly different structure and XRPD pattern, as well as different THz Raman spectra.

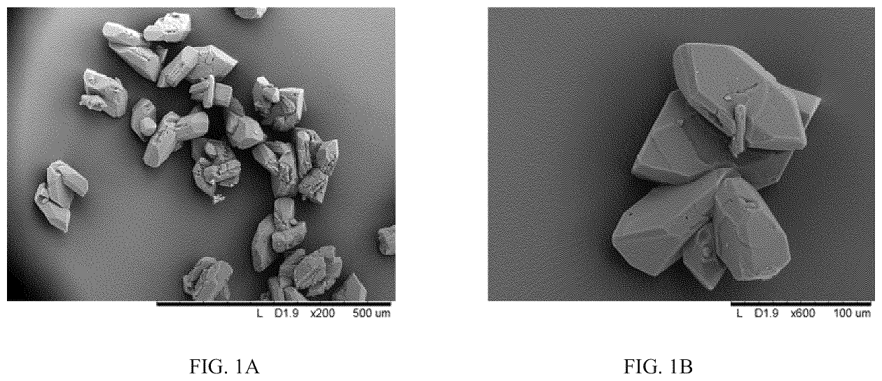

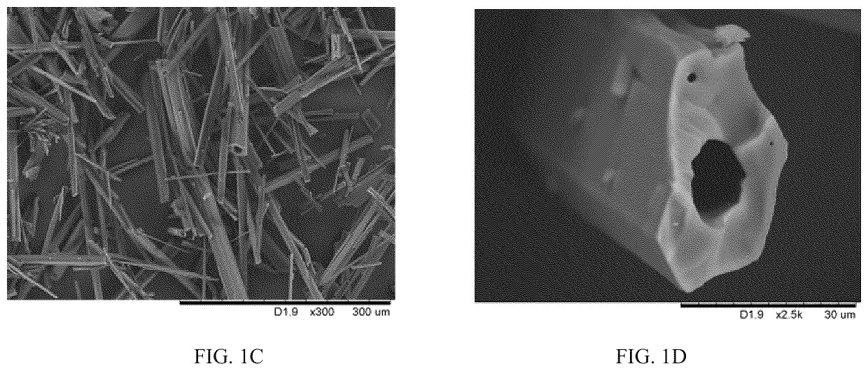

FIG. 1A and FIG. 1B present SEM images of (S)-Formula I HCl Form A crystals and FIG. 1C and FIG. 1D present SEM images of (S)-Formula I HCl Form B crystals. Form A was observed to form plate crystals and was determined by XRPD to have a monoclinic crystal system, while the Form B was observed to form hollow needle crystals and was determined by XRPD to have an orthorhombic crystal system. As isolated from conventional synthesis or salt conversion, (S)-Formula I HCl typically appears as a mixture of Forms A and B.

Form B was determined to be less thermodynamically stable than Form A, and can be converted by solid state conversion to Form A. The solid state conversion of the polymorph Form B needles to polymorph Form A blocks can be monitored by X-ray diffraction, and it was discovered unexpectedly that the visible morphology retains the needle shape while the crystal lattice changes to that of Form A.

### X-Ray Powder Diffraction (XRPD)

The XRPD pattern of FIG. 2A was obtained in transmission mode with a Stoe Stadi P (G.52.SYS.S072) with a Mythen1K detector, using Cu Kα radiation; with measurements in transmission mode; 40 kV and 40 mA tube power; a curved Ge monochromator detector; 0.02° 20 step size, with a 12 s step time, and a 1.5-50.5° 20 scanning range. The detector mode was set to: step scan with 1° 20 detector step and sample preparation was a 10 to 20 mg sample placed between two acetate foils and clamped in a Stoe transmission sample holder. Samples were rotated during the measurement.

The XRPD patterns of FIG. 2B and FIG. 2C were obtained with a Bruker 08 Advance, Cu Kα radiation (λ=1.54180 Å), with measurements in reflection mode; 40 kV/40 mA tube power; LynxEye detector, 0.02° step size in 20, using 37 s per step, and a 2.5°-50° 20 scanning range. The sample was prepared on silicon single crystal sample holders with 1.0 mm depth and was covered with Kapton foil. The sample was rotated during the measurement.

Further details of the crystal data and crystallographic data collection parameters are summarized in Table 1, and a listing of the peaks of the XRPD of FIG. 2A are listed in Table 2A, the peaks of the XRPD of FIG. 2B are listed in Table 2B, and the peaks of the XRPD of FIG. 2C are listed in Table 2C.

TABLE 1
(S)-Formula I HCl Form A and Form B Single Crystal Data and Collection Parameters

| | Form A, blocks | | Form B, needles | |
|---|---|---|---|---|
| Empirical formula | C₉H₁₄NOSCl | | C₉H₁₄NOSCl | |
| Molecular formula | [C₉H₁₄NOS]⁺[Cl]⁻ | | [C₉H₁₄NOS]⁺[Cl]⁻ | |
| Formula weight | 219.72 | | 219.72 | |
| Temperature | 100(2)K | | 100(2)K | |
| Wavelength | 1.54184Å | | 1.54184Å | |
| Crystal system | Monoclinic | | Orthorhombic | |
| Space group | P21 (#4) | | P212121 (#19) | |
| Unit cell dimensions | a = 9.1719(2) Å; | | a = 5.10405(5) Å; | |
| | α = 90°. | | α = 90°. | |
| | b = 11.2183(3)Å; | | b = 10.2114(1) Å; | |
| | β = 92.146(2)°. | | β = 90°. | |
| | c = 10.2092(2) Å; | | c = 20.5496(2) Å; | |
| | γ = 90°. | | γ = 90°. | |
| Volume | 1049.72(4) Å\hat{ }3 | | 1071.035(18) Å\hat{ }3 | |
| Z | 4 | | 4 | |
| Density (calculated) | 1.390 | Mg/m³ | 1.363 | Mg/m³ |
| Absorption coefficient | 4.765 | mm⁻¹ | 4.670 | mm⁻¹ |

| | | |
|---|---|---|
| F(000) | 464 | 464 |
| Crystal size | 0.0823 × 0.0529 × 0.0396 | 0.3254 × 0.0539 × 0.0366 |
| | mm³ | mm³ |
| Theta range for data collection | 4.33 to 76.58°. | 4.30 to 76.77°. |
| Index ranges | −11 <= h <= 10, −13 <= k <= 14, | −6 <= h <= 6, −12 <= k <= 12, |
| | −12 <= l <= 12 | −25 <= l <= 25 |
| Reflections collected | 11895 | 22468 |
| Independent reflections | 4211 [R(int) = 0.0362] | 2261 [R(int) = 0.0532] |
| Completeness to θ = 76.58° | 99.50% | 100.00% |
| Absorption correction | Analytical | Analytical |
| Max. and min. transmission | 0.860 and 0.776 | 0.864 and 0.435 |
| Refinement method | Full-matrix least-squares on | Full-matrix least-squares on |
| | F2 | F2 |
| Data/restraints/parameters | 4211/1/237 | 2261/3/136 |
| Goodness-of-fit on F2 | 1.041 | 1.085 |
| Final R indices [I > 2σ (I)] | R1 = 0.0264, wR2 = 0.0587 | R1 = 0.0270, wR2 = 0.0665 |
| R indices (all data) | R1 = 0.0289, wR2 = 0.0601 | R1 = 0.0291, wR2 = 0.0680 |
| Absolute structure parameter | −0.001(10) | −0.032(18) |
| Largest diff. peak and hole | 0.260 and −0.188 e.Å⁻³ | 0.329 and −0.573 e.Å⁻³ |

TABLE 2A
(S)- Formula I HCl Form A Single
Crystal XRPD (FIG. 2A) Peak List

| | Relative |
|---|---|
| 2-Theta | Height |
| 9.55 | 22.61 |
| 11.63 | 3.1 |
| 12.35 | 11.47 |
| 12.65 | 8.6 |
| 14.89 | 20.7 |
| 15.27 | 9.77 |
| 15.67 | 5.44 |
| 17.91 | 24.67 |
| 18.38 | 12.71 |
| 19.00 | 28.32 |
| 19.16 | 25.92 |
| 19.49 | 4.49 |
| 20.19 | 27.17 |
| 20.48 | 33.87 |
| 20.72 | 15.32 |
| 24.84 | 19.16 |
| 25.11 | 100 |
| 25.57 | 76.5 |
| 26.11 | 3.05 |
| 26.56 | 2.56 |
| 26.86 | 6.74 |
| 27.07 | 16.04 |
| 27.24 | 4.78 |
| 27.52 | 2.28 |
| 28.60 | 2.32 |
| 28.91 | 5.9 |
| 29.22 | 2.58 |
| 29.98 | 2.52 |
| 30.55 | 3.87 |
| 30.81 | 6.64 |
| 31.63 | 23.29 |
| 32.00 | 2.86 |
| 32.84 | 4.04 |
| 33.05 | 5.83 |
| 34.37 | 1.81 |
| 34.98 | 2.25 |
| 35.41 | 2.97 |
| 36.61 | 1.82 |
| 37.02 | 3.83 |
| 37.59 | 1.99 |
| 38.46 | 1.71 |
| 39.47 | 4.25 |

TABLE 2B
(S)-Formula I HCl Form A Single
Crystal XRPD (FIG. 2B) Peak List

| | Relative |
|---|---|
| 2-Theta | Height |
| 9.59 | 27.73 |
| 11.70 | 3.44 |
| 12.35 | 17.88 |
| 12.69 | 11.1 |
| 13.12 | 3.52 |
| 14.93 | 22.25 |
| 15.31 | 11.24 |
| 15.71 | 4.04 |
| 17.28 | 7.28 |
| 17.95 | 18.38 |
| 18.41 | 14.03 |
| 19.16 | 91.74 |
| 19.53 | 9.85 |
| 20.23 | 31.95 |
| 20.51 | 42.51 |
| 20.76 | 27.67 |
| 21.60 | 3.41 |
| 22.25 | 3.33 |
| 22.77 | 3.87 |
| 24.82 | 77.41 |
| 25.14 | 100 |
| 25.59 | 82 |
| 26.13 | 7.25 |
| 26.58 | 6.46 |
| 27.10 | 19.49 |
| 27.55 | 6.3 |
| 28.87 | 27.5 |
| 29.24 | 5.41 |
| 29.99 | 8.48 |
| 30.55 | 8.74 |
| 30.83 | 12.69 |
| 31.63 | 24.78 |
| 32.02 | 5.46 |
| 33.03 | 12.75 |
| 34.31 | 5.42 |
| 34.93 | 9.12 |
| 35.45 | 5.72 |
| 35.99 | 4.93 |
| 36.68 | 6.56 |
| 37.58 | 8.48 |
| 38.49 | 4.42 |
| 39.47 | 6.41 |

TABLE 2C
(S)-Formula I HCl Form B Single
Crystal XRPD (FIG. 2C) Peak List

| | Relative |
|---|---|
| 2-Theta | Height |
| 8.54 | 9.1 |
| 8.89 | 0.3 |
| 11.76 | 0.8 |
| 12.12 | 21.6 |
| 12.45 | 0.2 |
| 15.46 | 1.8 |
| 17.12 | 100 |
| 17.48 | 1.7 |
| 17.82 | 2.6 |
| 18.32 | 0.5 |
| 19.18 | 14.2 |
| 21.56 | 0.5 |
| 23.16 | 9.5 |
| 24.80 | 0.9 |
| 25.80 | 6.6 |
| 26.20 | 0.2 |
| 27.26 | 2.1 |
| 27.62 | 0.6 |
| 29.06 | 1.2 |
| 31.50 | 15.4 |
| 31.81 | 0.4 |
| 32.42 | 0.5 |
| 33.87 | 0.6 |
| 34.68 | 1 |
| 35.00 | 1.9 |
| 35.76 | 1.6 |
| 36.94 | 0.9 |
| 37.24 | 0.4 |
| 39.28 | 0.1 |
| 40.00 | 0.7 |
| 40.20 | 1.1 |
| 43.08 | 5.8 |
| 43.74 | 2 |
| 44.60 | 0.6 |

### Raman and THz Raman Spectra

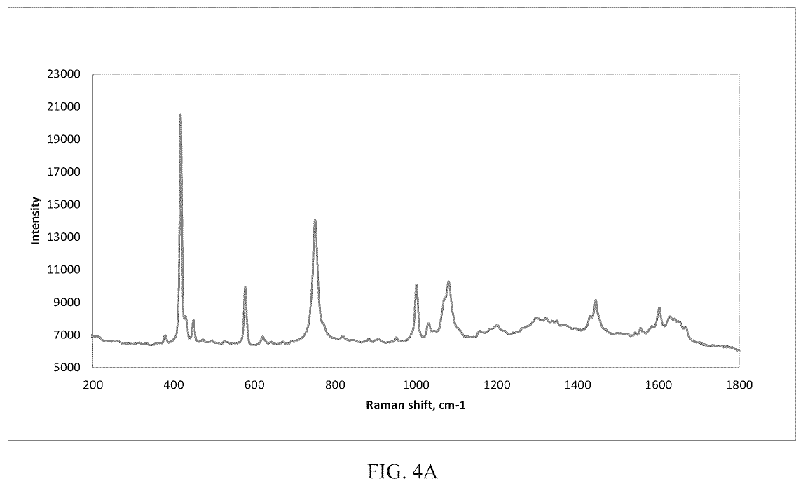

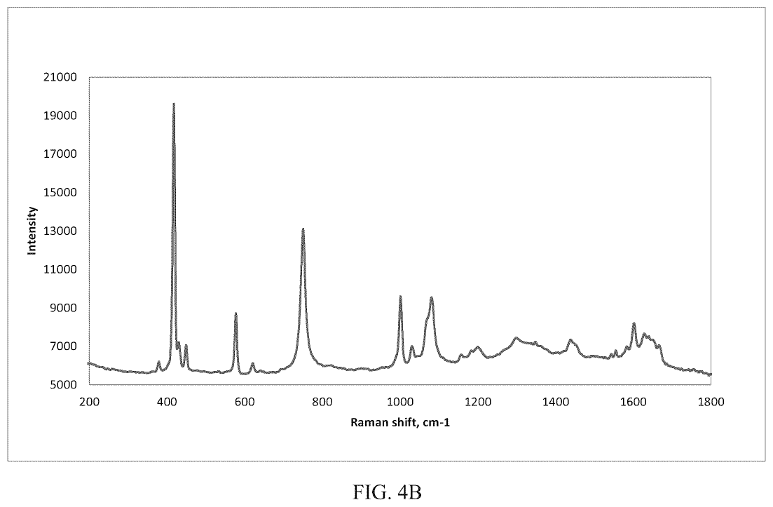

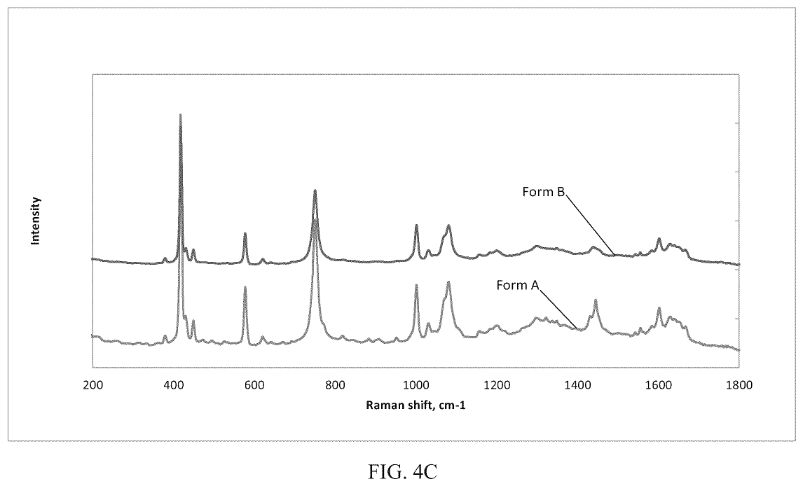

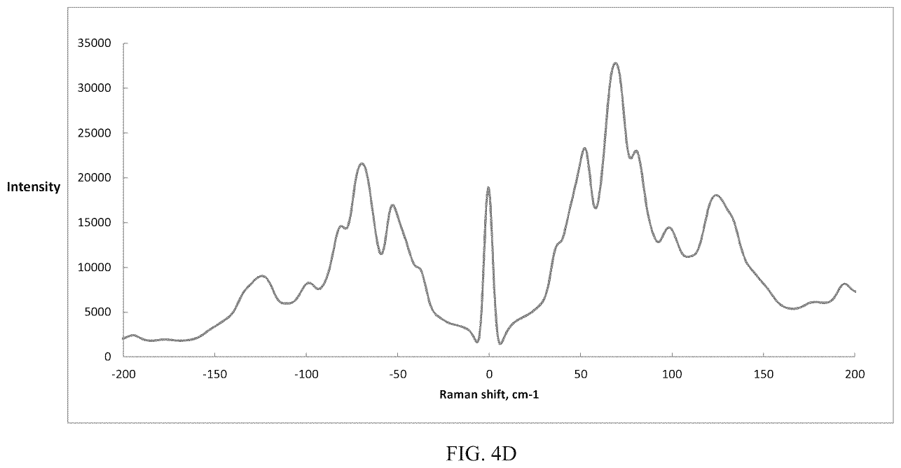

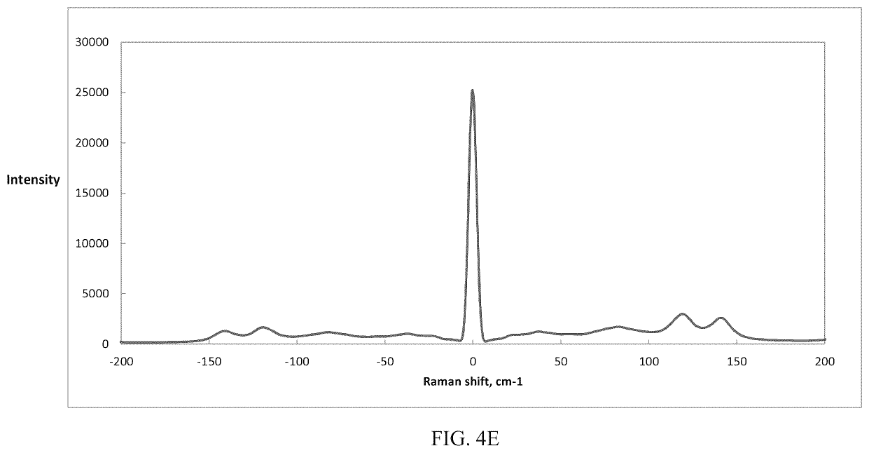

The Raman and THz Raman spectroscopic analysis was performed using a Kaiser Raman RXN-Hybrid-785 system with laser wavelength 785 nm, with a spectral coverage of +100 cm⁻¹ to +1875 cm⁻¹ for the Raman spectra and a spectral coverage of −200 cm⁻¹ to +200 cm⁻¹ for the Tz Raman spectra; spectral resolution was 4 cm⁻¹. The Raman spectra of FIG. 4A, FIG. 4B and FIG. 4C were collected with the regular immerse Raman probe, and the THz Raman spectra of FIG. 4D and FIG. 4E were collected with the THz-Raman® Probe.

Referring to FIG. 4A and FIG. 4C, (S)-Formula I HCl Form A crystals were used as a powder and the spectra taken in a dark chamber. Referring to FIG. 4B and FIG. 4C, (S)-Formula I HCl Form B crystals were freshly generated by dissolving Form A crystals in isopropanol and then rotary evaporating off the solvent, then the Form B crystals were used as a powder and the spectra taken in a dark chamber. A listing of various peaks in the spectra of FIG. 4A are provided in Table 3A, and various peaks in the spectra of FIG. 4B are provided in Table 3B.

Referring to FIG. 4D, (S)-Formula I HCl Form A crystals were suspended in isopropanol at room temperature and the THz-Raman® Probe used to take the spectra in the suspension. Referring to FIG. 4E, (S)-Formula I HCl Form B crystals were generated by the reverse dumping addition of freebase (S)-Formula I to the HCl solution, and THz-Raman® Probe immediately used to take the spectra in suspension.

Both the Raman spectra and THz Raman spectra were obtained using: (a) cosmic ray filtering' and (b) baseline correction and smoothing to obtain interpretable data when necessary; and for the THz Raman spectra background subtraction of a well filled with IPA collected with the same conditions.

TABLE 3A
(S)-Formula I HCl Form A Raman Spectra (FIG. 4A) Peak List

| | Relative Peak |
|---|---|
| Raman shift, cm⁻¹ | Height |
| 378.9 | 31.53 |
| 417.6 | 100.00 |
| 430.2 | 36.77 |
| 448.8 | 35.97 |
| 576.9 | 44.40 |
| 620.7 | 31.18 |
| 750.0 | 66.84 |
| 1001.1 | 48.84 |
| 1030.8 | 35.65 |
| 1080.9 | 48.59 |
| 1439.1 | 37.41 |
| 1602.3 | 41.81 |

TABLE 3B
(S)-Formula I HCl Form B Raman Spectra (FIG. 4B) Peak List

| | Relative Peak |
|---|---|
| Raman shift, cm⁻¹ | Height |
| 378.9 | 33.95 |
| 417.6 | 100.00 |
| 429.6 | 39.79 |
| 448.8 | 38.43 |
| 577.2 | 48.47 |
| 620.4 | 33.63 |
| 750.3 | 68.58 |
| 1001.1 | 49.14 |
| 1030.8 | 37.64 |
| 1080.6 | 50.10 |
| 1445.1 | 44.63 |

Referring to FIG. 4D and FIG. 4E, the THz Raman spectra of the two polymorphs is distinctly different. For example, in various embodiments, the THZ Raman spectra of the Raman peak of Form B at 1162 cm⁻¹ and the THZ Raman spectra of the Raman peak of Form A at 1089 cm⁻¹ can be used to distinguish these polymorphs.

Crystalline (S)-Formula I HCl Forms A and (S)-Formula I HCl Form B exhibit different properties and different “fingerprints”. Various measurements presented herein on these polymorphs are summarized in Table 4.

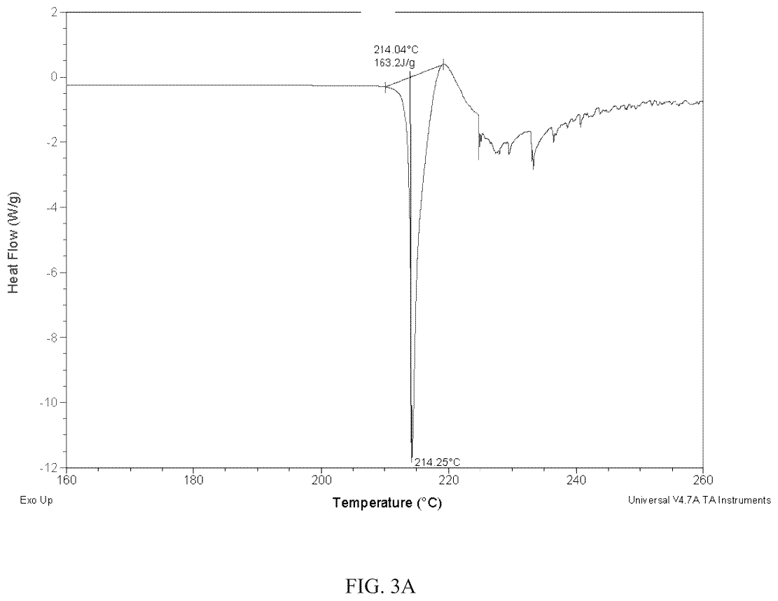

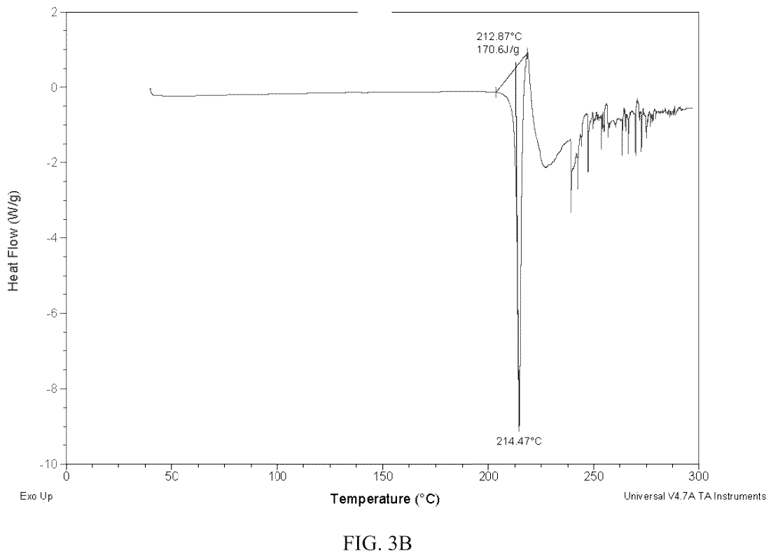

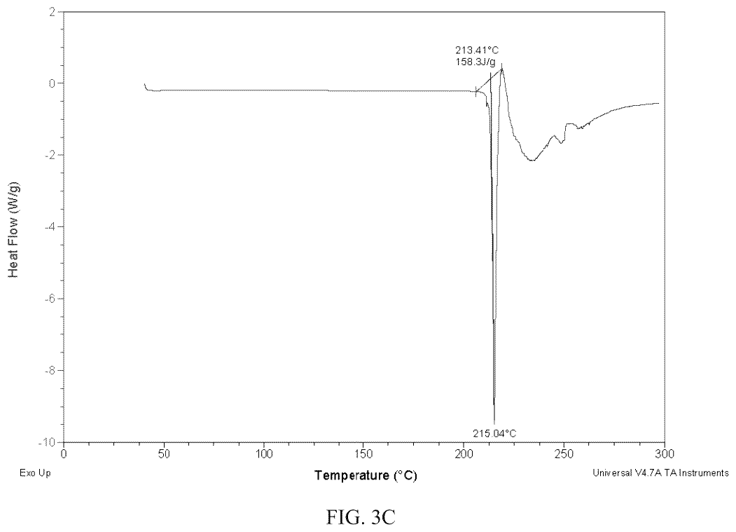

TABLE 4
Summary of Measurements

| | Form A | Form B |
|---|---|---|
| SEM Image | FIG. 1A; FIG. 1B | FIG. 1C; FIG. 1D |
| XRPD Pattern | FIG. 2A; FIG. 2B | FIG. 2C |
| DSC Thermograph | FIG. 3A | FIG. 3B; FIG. 3C |
| Raman | FIG. 4A | FIG. 4B |
| THz Raman | FIG. 4D | FIG. 4E |

In some embodiments, provided is a crystalline form of (S)-Formula I HCl characterized by an XRPD pattern comprising peaks, in terms of 2-theta, at 9.6±0.2°, 14.9±0.2°, 20.5±0.2°, and 25.1±0.2°, and a DSC thermogram having a peak at 214±2° C.

In some embodiments, provided is a crystalline form of (S)-Formula I HCl characterized by an XRPD pattern comprising peaks, in terms of 2-theta, at 9.6±0.2°, 14.9±0.2°, 20.5±0.2°, and 25.1±0.2°, and a differential scanning calorimetry thermogram substantially in accord with FIG. 3A.

In some embodiments, provided is a crystalline form of (S)-Formula I HCl characterized by an XRPD pattern comprising peaks, in terms of 2-theta, at 9.6±0.2°, 14.9±0.2°, 20.5±0.2°, and 25.1±0.2°, and a Raman spectra substantially in accord with FIG. 4A and/or a THz Raman spectra substantially in accord with FIG. 4D.

In some embodiments, provided is a crystalline form of (S)-Formula I HCl characterized by an XRPD pattern comprising peaks, in terms of 2-theta, at 8.6±0.2°, 17.2±0.2°, and 25.9±0.2°, and a DSC thermogram having a peak at 215±2° C.

In some embodiments, provided is a crystalline form of (S)-Formula I HCl characterized by an XRPD pattern comprising peaks, in terms of 2-theta, at 8.6±0.2°, 17.2±0.2°, and 25.9±0.2°, and a differential scanning calorimetry thermogram substantially in accord with FIG. 3B or FIG. 3C.

In some embodiments, provided is a crystalline form of (S)-Formula I HCl characterized by an XRPD pattern comprising peaks, in terms of 2-theta, at 8.6±0.2°, 17.2±0.2°, and 25.9±0.2°, and a Raman spectra substantially in accord with FIG. 4B and/or a THz Raman spectra substantially in accord with FIG. 4E.

In some embodiments, provided is a crystalline form of (S)-Formula I HCl that is the substantially non-hygroscopic. In various embodiments, the present inventions provide a crystalline (S)-Formula I HCl of Form A that has a maximum mass change of less than about 1%, less than about 0.5%, less than about 0.3%, less than about 0.2%, or less than about 0.1% in water sorption isotherms as measured by dynamic vapor sorption (DVS), at 25° C. scanned over 0 to 90% relative humidity.

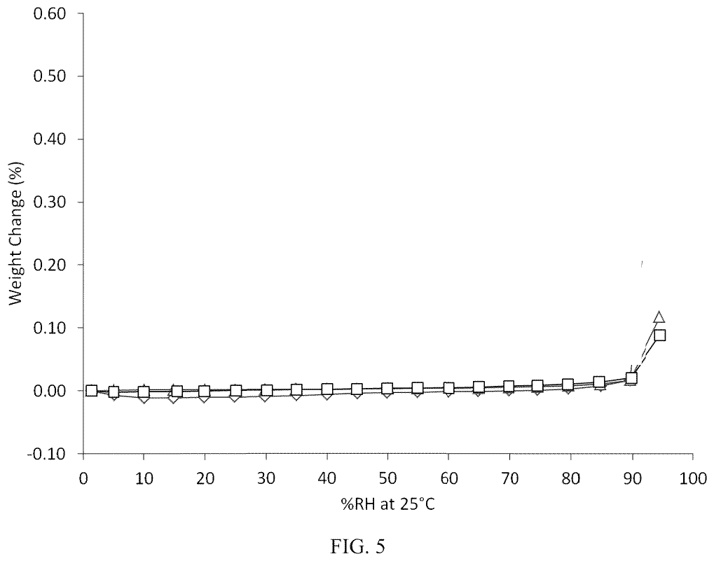

FIG. 5 and Table 5 present DVS water sorption isotherms for crystalline (S)-Formula I HCl of Form A. The water sorption isotherms were generated using a VTI SGA-100 dynamic vapor sorption analyzer. Samples were dried pre-analysis at 25° C. with equilibrium criteria of 0.0000 wt % changes in 5 minutes or a maximum of 180 minutes. Isotherm equilibrium criteria were the lesser of 0.01 wt % change in 5 minutes or 180 minutes at each relative humidity (RH) step. Temperature was fixed at 25° C. and the relative humidity steps (5% to 95% to 5%) were in 5% increments. Initial sample size ranged from 41 to 47 mg.

FIG. 5 presents DVS water sorption for two different lots of crystalline (S)-Formula I HCl of Form A, and Table 5 lists the data plotted in FIG. 5. As can be seen, crystalline (S)-Formula I HCl Form A is substantially non-hygroscopic, exhibiting a maximum mass change of only 0.2% at 95% relative humidity (RH), and less than a 0.1% mass change at 90% RH and below.

TABLE 5
(S)-Formula I HCl Form A DVS Water
Sorption Isotherms of FIG. 5

| | Lot 1 (square symbols) | | Lot 2 (upright triangle symbols) | |
|---|---|---|---|---|
| Relative | Change | Elapse | Change | Elapse |
| Humidity | Mass | Time | Mass | Time |
| (%) | (%) | (min) | (%) | (min) |
| 1 | 0.000 | 155.6 | 0.000 | 41.6 |
| 5 | −0.002 | 329.5 | 0.001 | 52.2 |
| 10 | −0.002 | 416.5 | 0.001 | 61.2 |
| 15 | −0.001 | 425.0 | 0.001 | 69.7 |
| 20 | −0.001 | 434.5 | 0.001 | 81.7 |
| 25 | 0.000 | 454.0 | 0.001 | 93.7 |
| 30 | 0.001 | 466.0 | 0.001 | 105.2 |
| 35 | 0.001 | 479.5 | 0.002 | 118.2 |
| 40 | 0.002 | 491.0 | 0.002 | 129.7 |
| 45 | 0.003 | 500.6 | 0.003 | 139.2 |
| 50 | 0.003 | 511.6 | 0.003 | 150.2 |
| 55 | 0.004 | 520.6 | 0.003 | 159.2 |
| 60 | 0.005 | 531.6 | 0.004 | 170.2 |
| 65 | 0.006 | 542.6 | 0.005 | 181.2 |
| 70 | 0.007 | 553.6 | 0.005 | 192.2 |
| 75 | 0.008 | 562.6 | 0.006 | 201.2 |
| 80 | 0.010 | 571.6 | 0.008 | 210.2 |
| 85 | 0.014 | 580.6 | 0.011 | 219.2 |
| 90 | 0.021 | 589.6 | 0.017 | 228.2 |
| 95 | 0.088 | 616.0 | 0.117 | 260.2 |

In some embodiments, provided is a crystalline form of (S)-Formula I HCl characterized by an XRPD pattern comprising peaks, in terms of 2-theta, at 9.6±0.2°, 14.9±0.2°, 20.5±0.2°, and 25.1±0.2°; in some embodiments, further characterized by peaks at 20.2±0.2° and 20.8±0.2°; and in some embodiments, further characterized by two or more prominent peaks in its XRPD pattern selected from those at 17.9±0.2°, 24.8±0.2° and 27.1±0.2°, in terms of 2-theta. In some embodiments, provided is a crystalline form of (S)-Formula I HCl characterized by an XRPD pattern substantially in accord with FIG. 2B.

In some embodiments, provided is a crystalline form of (S)-Formula I HCl of Form A characterized by the following properties, an XRPD pattern comprising peaks, in terms of 2-theta, at 9.6±0.2°, 14.9±0.2°, 20.5±0.2°, and 25.1±0.2°, a melting point of 214±2° C., a chiral purity of greater than about 99%, a chemical purity greater than about 99%, a residual solvent content of less than about 8000 ppm, and is substantially non-hygroscopic.

In some embodiments, provided is a crystalline form of (S)-Formula I HCl characterized by the following properties, an XRPD pattern comprising peaks, in terms of 2-theta, at 9.6±0.2°, 14.9±0.2°, 20.5±0.2°, and 25.1±0.2° and one or more of the following:
(a) the powder x-ray diffraction pattern further comprising peaks, in terms of 2-theta, at 20.2±0.2° and 20.8±0.2°;
(b) the powder x-ray diffraction pattern further comprising a prominent peak, in terms of 2-theta, at two of more of 17.9±0.2°, 24.8±0.2° and 27.1±0.2°;
(c) a melting point of 214±2° C.;
(d) a differential scanning calorimetry thermogram comprising a peak at 214±2° C.;
(e) a differential scanning calorimetry thermogram substantially in accord with FIG. 3A;
(f) a Raman spectra substantially in accord with FIG. 4A, a THz Raman spectra substantially in accord with FIG. 4D, or both;
(g) a chiral purity of greater than about: (i) 90%, (ii) 95%, (iii) 97%, (iv) 99%, (v) 99.5%, (vi) 99.7%, or (vii) 99.9%;
(h) a chemical purity of greater than about: (i) 80%, (ii) 90%, (iii) 95%, (iv) 97%, (v) 99%, (vi) 99.5%, (vii) 99.7%, or (viii) 99.9%;
(i) residual solvents present in an amount less than about: (i) 8000 ppm, (ii) 6000 ppm, (iii) 4000 ppm, (iv) 2000 ppm, (v) 1000 ppm, (vi) 800 ppm, or 500 ppm;
(j) as measured by dynamic vapor sorption (DVS), at 25° C. scanned over 0 to 95% relative humidity, a maximum mass change in water sorption isotherms of less than about (i) 2%, (ii) 1%, (iii) 0.5%, (iv) 0.4%, (v) 0.3%, (vi) 0.2%, or (vii) 0.1%; and
(k) as measured by dynamic vapor sorption (DVS), at 25° C. scanned over 0 to 90% relative humidity, a maximum mass change in water sorption isotherms of less than about (i) 1%, (ii) 0.5%, (iii) 0.4%, (iv) 0.3%, (v) 0.2%, or (vi) 0.1%; nd preferably less than about 0.2%.

In some embodiments, provided is a crystalline form of (S)-Formula I HCl characterized by an XRPD pattern comprising peaks, in terms of 2-theta, at 8.6±0.2°, 17.2±0.2°, and 25.9±0.2°; and in some embodiments, further characterized by peaks in its XRPD pattern selected at, 23.2±0.2°, and 31.5±0.2°, in terms of 2-theta. In some embodiments, provided is a crystalline form of (S)-Formula I HCl characterized by an XRPD pattern substantially in accord with FIG. 2C.

In some embodiments, provided is a crystalline form of (S)-Formula I HCl of Form B characterized by the following properties, an XRPD pattern comprising peaks, in terms of 2-theta, at 8.6±0.2°, 17.2±0.2°, and 25.9±0.2°, and a melting point of 215±2° C.

In some embodiments, provided is a crystalline form of (S)-Formula I HCl characterized by the following properties, an XRPD pattern comprising peaks, in terms of 2-theta, at 8.6±0.2°, 17.2±0.2°, and 25.9±0.2° and one or more of the following:
(a) the powder x-ray diffraction pattern further comprising peaks, in terms of 2-theta, at 23.2±0.2°, and 31.5±0.2°;
(b) a melting point of 215±2° C.;
(c) a differential scanning calorimetry thermogram comprising a peak at 215±2° C.;
(d) a differential scanning calorimetry thermogram substantially in accord with FIG. 3B or 3C;
(e) a Raman spectra substantially in accord with FIG. 4B, a THz Raman spectra substantially in accord with FIG. 4E, or both;
(f) a chiral purity of greater than about: (i) 90%, (ii) 95%, (iii) 97%, (iv) 99%, (v) 99.5%, (vi) 99.7%, or (vii) 99.9%;
(g) a chemical purity of greater than about: (i) 80%, (ii) 90%, (iii) 95%, (iv) 97%, (v) 99%, (vi) 99.5%, (vii) 99.7%, or (viii) 99.9%; and
(h) residual solvents present in an amount less than about: (i) 8000 ppm, (ii) 6000 ppm, (iii) 4000 ppm, (iv) 2000 ppm, (v) 1000 ppm, (vi) 800 ppm, or 500 ppm; and
In some embodiments, provided are methods for preparing (S)-Formula I HCl as crystalline Form A. In some embodiments, the method of making crystalline (S)-Formula I HCl Form A begins with (S)-Formula I. In some embodiments, the method of making crystalline (S)-Formula I HCl Form A begins with substantially racemic Formula I.

In some embodiments, provided are methods for preparing crystalline (S)-Formula I HCl Form A with various particle size distributions.

Example 1 provides and illustrates various embodiments of methods of making (S)-Formula I HCl Form A. Example 2 provides and illustrates various embodiments of methods of making various particle size distributions of (S)-Formula I HCl Form A.

A synthesis of racemic Formula I

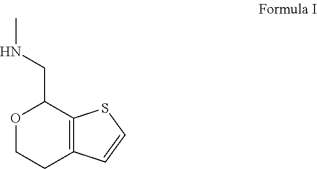

is disclosed in U.S. Pat. No. 8,710,245, which is hereby incorporate by reference in its entirety. In U.S. Pat. No. 8,710,245, the racemate is resolved into the single (R) and (S) enantiomers:

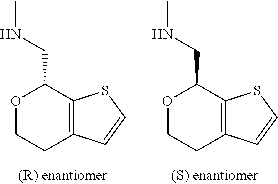

by column chromatography. The free base of (S)-Formula I is a yellow oil that degrades over time when exposed to air.

In some embodiments of the methods disclosed herein, the balance between crystalline Form A and Form B is driven to substantially pure crystalline Form A by the controlled addition of a solution of between about 5% to about 10% HCl in isopropanol into a solution of (S)-Formula I free base in isopropanol at a temperature between 20° C. and 60° C., preferably about 40° C. In some embodiments, the controlled addition is carried out as a logarithmic-like addition wherein the HCl solution is added slowly at first and the rate is steadily increased. The HCl addition rate, in various embodiments, 10% of the HCl solution is added over a first time period of between about 10 minutes and about 90 minutes, 30% of the HCl solution is added over a second time period of between about 10 minutes and about 90 minutes, and the remainder of the HCl solution is added over a third time period of between about 10 minutes and about 90 minutes.

In some embodiments, the slow addition of acid solution (e.g., slower supersaturation rate) with a logarithmic-like addition profile (examples include, but are not limited to, a the Mullin-Nyvlt type addition profile, see, e.g., J. W. Mullin and J. Nyvlt, Chem Eng Sci. 1971; 26:3, 369-377;), higher operation temperature, lower concentration of starting freebase solution, and higher water content of the crystallization mixture, favor the generation of large crystals of (S)-Formula I HCl Form A; whereas lower operation temperature, higher concentration of starting freebase solution, and lower water content of the crystallization mixture, favor the generation of smaller crystals of (S)-Formula I HCl Form A. It is to be understood, that mean, average and/or median particle size is generally not the sole determinant of a desirable PSD, rather, the width of a PSD is often of importance.

### Particle Size Distribution (PSD)

In some embodiments, provided are methods of modulating the particle size distribution of crystalline (S)-Formula I HCl and in particular of crystalline (S)-Formula I HCl Form A, into a desired range, for example, a PSD favorable for compressing tablets and/or providing good solution kinetics. In some embodiments, the particle size distribution of the (S)-Formula I HCl can be modulated by: (i) the addition rate of HCl during the formation of (S)-Formula I HCl (e.g. Step 4b in Scheme 4); (ii) the concentration of (S)-Formula I freebase in the solution prior to HCl addition (e.g. Compound F concentration in Scheme 4 between Steps 4a and 4b); (iii) the temperature of the solution during HCl addition; (iv) the water content of the crystallization mixture; and (v) the reaction process.

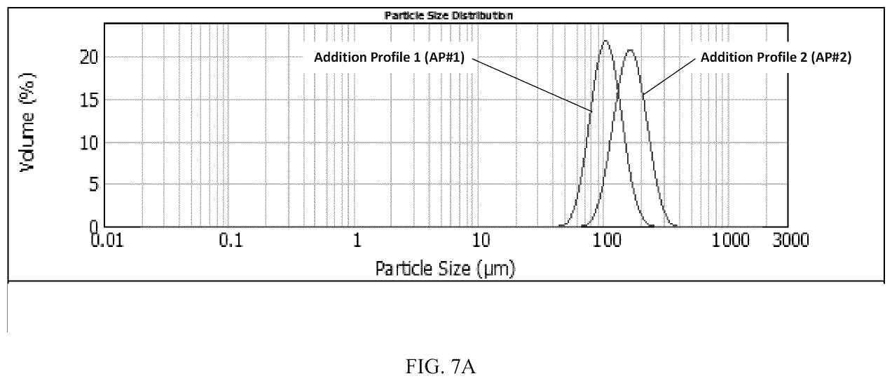

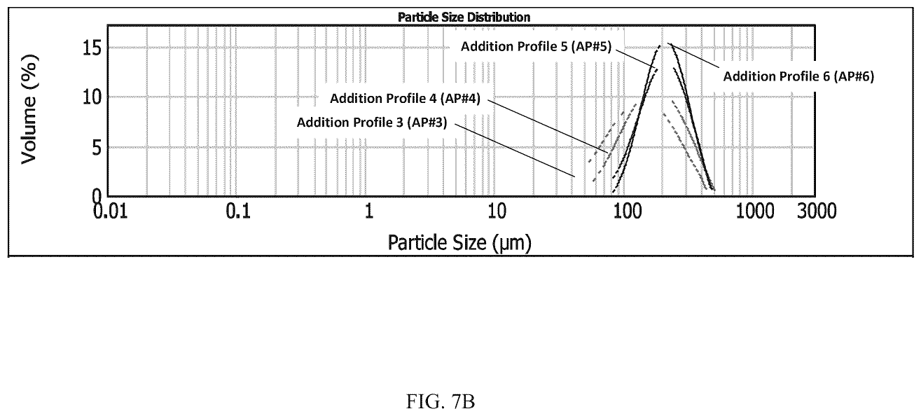

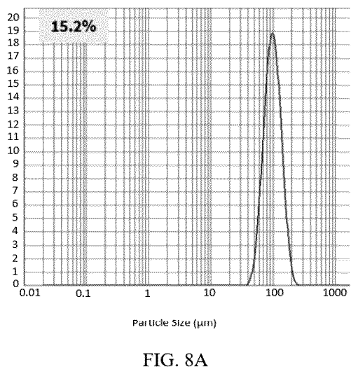

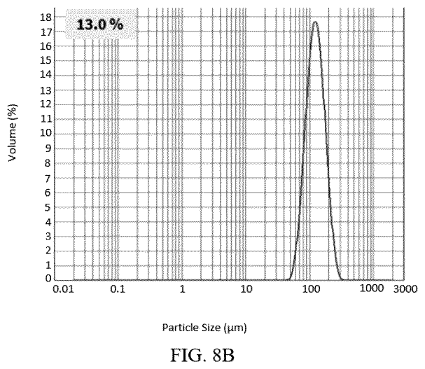

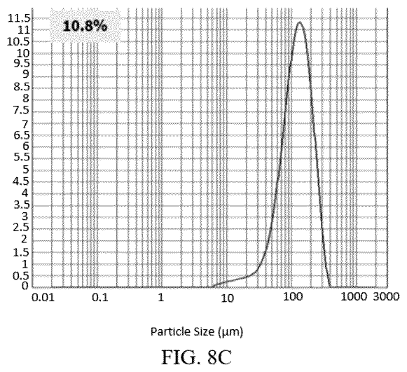

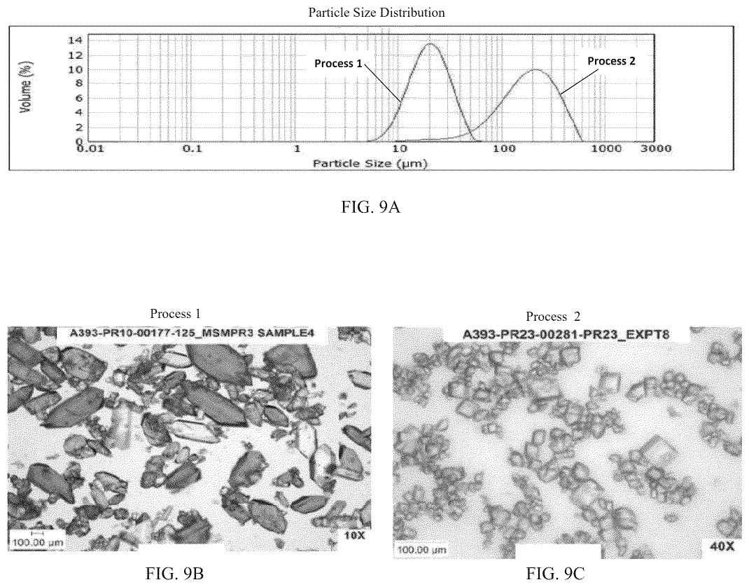

Referring to FIG. 7A, FIG. 7B, FIG. 8A, FIG. 8B, FIG. 8C, and FIG. 9A, presented are various PSD data for (S)-Formula I HCl Form A, obtained under various conditions as further discussed in Example 2. The PSD data of FIG. 7A, FIG. 7B, FIG. 8A, FIG. 8B and FIG. 8C was obtained by a laser diffraction particle sizing technique using a Malvern Mastersizer 2000 analyzer instrument and the PSD data of FIG. 9A by a laser diffraction particle sizing technique using a Horiba LA-920 instrument, and all data is presented as volume % as a function of particle size.

In some embodiments, the PSD of crystalline (S)-Formula I HCl Form A can be affected by the supersaturation generation rate (e.g. controlled by the dosing profile of the HCl solution Step 4b of Scheme 4), operation temperature, water content, and reaction process (e.g. mixing, sonication, etc.). For example, in some embodiments, sonication during addition of HCl to form (S)-Formula I HCl (e.g. Step 4b in Scheme 4) can dramatically decrease the final (S)-Formula I HCl Form A crystal size (e.g. D50=20 to 30 μm) by promoting the nucleation over the course of addition of HCl.

In some embodiments of the reactive-crystallization of (S)-Formula I HCl, the supersaturation generation rate can be directly controlled by the HCl solution addition rate; faster dosing (HCl addition) favoring the formation of smaller crystals and slower dosing favoring the formation of larger crystals. However, faster addition results in wider PSD distributions.

In some embodiments, operational temperature can be used to affect the kinetic behavior for nucleation and crystal growth, as well as solubility. Higher temperatures increase mean crystal size and width of the PSD.

In some embodiments, starting (S)-(−)-Formula I freebase concentration prior to reactive recrystallization can be used to affect the kinetic behavior for nucleation and crystal growth. In some embodiments, a higher starting (S)-(−)-Formula I freebase concentration will decrease both the median particle size and the width of the PSD.

In some embodiments, alkyl alcohols of 4 carbons or less, including but not limited to, n-propanol, isopropanol, and n-butanol can be used.

In some embodiments, the (S)-Formula I free base is dissolved in a solvent system comprising from 90% to 100% isopropanol. In some embodiments, the solvent system is 90% to 99% isopropanol and the remainder is water. In some embodiments the solvent system is 93% to 97% isopropanol and the remainder is water. In some embodiments, the solvent system is >99% isopropanol. The presence of water, in some embodiments, of up to about 5% leads to crystals of (S)-Formula I HCl polymorph Form A that are more cubic than hexagonal in morphology. In some embodiments, the methods disclosed herein provide for crystalline (S)-Formula I HCl Form A with increased cubic morphology. In some embodiments of the composition, medicaments and formulations disclosed herein, crystalline (S)-Formula I HCl Form A with increased cubic morphology are preferred as being more flowable than the hexagonal morphology, and as possessing advantages in formation of certain solid oral dosage forms (e.g., in certain tableting operations).

In Example 1, the hydrogen chloride in isopropanol was prepared at 6% by weight, but could be employed in other concentrations: for example, in some embodiments from about 4% to about 10%. In some embodiments, the HCl in an alkyl alcohol of 4 carbons or less, e.g. isopropanol, can be added in ratios from 1.0 to 1 up to 1.2 to 1 stoichiometry based on the amine in (S)-Formula I.

The concentration of (S)-Formula I free base in the alkyl alcohol of 4 carbons or less, e.g. isopropanol, was observed to be operable over a wide range. In some embodiments, the concentration of (S)-Formula I free base solution is between about 5.0% to 25.0% by weight %, and preferably between about 10% and about 15%. In some embodiments, the concentration of (S)-Formula I free base solution is about 10.0%, about 11.0%, about 13.0%, or about 15.0% by weight %.

One of ordinary skill in the art would understand that very dilute solutions of (S)-Formula I free base are likely to produce lower yields because of the finite solubility of (S)-Formula I HCl in alkyl alcohols of 4 carbons or less, e.g. isopropanol.

The particle size distribution of crystalline (S)-Formula I HCl Form A can be controlled by the balance among the reactant addition rate, local and global supersaturation, mass transfer and crystal surface area. The slow addition of acid solution, for example, with a Mullin-Nyvlt-like addition profile, higher operation temperature, lower concentration of starting freebase solution, presence of water in the solvent system, seeding favors the formation of the larger crystalline (S)-Formula I HCl Form A crystals, and sonication during supersaturation favors the formation of the smaller crystalline (S)-Formula I HCl Form A crystals.

In some embodiments, provided are compounds comprising (S)-Formula I HCl Form A crystals having a particle size distribution (when measured by laser diffraction, for example, as set forth in Example 2) with a median (D50) between about 15 μm to about 30 μm, a D10 greater than about 10 μm and a D90 less than about 40 μm; and preferably with a D50 between about 20 μm to about 30 μm.

In some embodiments, provided are compounds comprising (S)-Formula I HCl Form A crystals having a particle size distribution (when measured by laser diffraction, for example, as set forth in Example 2) with a median (D50) between about 15 μm to about 30 μm, (and preferably between about 20 μm to about 30 μm), and a span less than about 1.75, less than about 1.5, less than about 1, or less than about 0.8.

In some embodiments, provided are compounds comprising (S)-Formula I HCl Form A crystals having a particle size distribution (when measured by laser diffraction, for example, as set forth in Example 2) with a median (D50) between about 100 μm to about 135 μm (and preferably a D50 between about 100 μm to about 110 μm), a D10 greater than about 60 μm and a D90 less than about 165 μm; and preferably with a D10 greater than about 70 μm and a D90 less than about 150 μm.

In some embodiments, provided are compounds comprising (S)-Formula I HCl Form A crystals having a particle size distribution (when measured by laser diffraction, for example, as set forth in Example 2) with a median (D50) between about 100 μm to about 135 μm (and preferably a D50 between about 100 μm to about 110 μm), and a span less than about 1.75, less than about 1.5, less than about 1, or less than about 0.8.

In some embodiments, provided are compounds comprising (S)-Formula I HCl Form A crystals having a particle size distribution (when measured by laser diffraction, for example, as set forth in Example 2) with a median (D50) between about 135 μm to about 180 μm (and preferably a D50 between about 160 μm to about 170 μm), a D10 greater than about 100 μm and a D90 less than about 250 μm; and preferably with a D10 greater than about 110 μm and a D90 less than about 230 μm.

In some embodiments, provided are compounds comprising (S)-Formula I HCl Form A crystals having a particle size distribution (when measured by laser diffraction, for example, as set forth in Example 2) with a median (D50) between about 135 μm to about 180 μm (and preferably a D50 between about 160 μm to about 170 μm), and a span less than about 1.75, less than about 1.5, less than about 1, or less than about 0.8.

In some embodiments, provided are compounds comprising (S)-Formula I HCl Form A crystals having a particle size distribution (when measured by laser diffraction, for example, as set forth in Example 2) with a median (D50) between about 180 μm to about 230 μm (and preferably a D50 between about 190 μm to about 220 μm), a D10 greater than about 110 μm and a D90 less than about 350 μm; and preferably with a D10 greater than about 120 μm and a D90 less than about 340 μm.

In some embodiments, provided are compounds comprising (S)-Formula I HCl Form A crystals having a particle size distribution (when measured by laser diffraction, for example, as set forth in Example 2) with a median (D50) between about 180 μm to about 230 μm (and preferably a D50 between about 190 μm to about 220 μm), and a span less than about 1.75, less than about 1.5, less than about 1, or less than about 0.8.

In some embodiments, the methods disclosed herein provide for (S)-Formula I HCl Form A crystals having a PSD (when measured by laser diffraction, for example, as set forth in Example 2) with a median (D50) between about 15 μm to about 30 μm, a D10 greater than about 10 μm and a D90 less than about 40 μm; and preferably with a D50 between about 20 μm to about 30 μm, a D10 greater than about 10 μm and a D90 less than about 40 μm; where the methods comprise sonication during a step of supersaturation of a freebase solution of (S)-Formula I to form (S)-Formula I HCl.

In various embodiments, the methods disclosed herein provide for (S)-Formula I HCl Form A crystals having a PSD (when measured by laser diffraction, for example, as set forth in Example 2) with a median (D50), in some embodiments, between about 100 μm to about 230 μm, between about 100 μm to about 135 μm, between about 135 μm to about 180 μm, or between about 180 μm to about 230 μm; and having a span less than about 1.75, less than about 1.5, less than about 1, or less than about 0.8; where the methods comprise using a logarithmic-like addition of HCl during the reactive-recrystallization of (S)-Formula I to form (S)-Formula I HCl. In some embodiments, the logarithmic-like addition comprises addition of between about 10% to about 15% of an HCl solution over a first time period, addition of about 30% to about 40% of the HCl solution over a second time period after the first time period, and addition of the remainder (between about 45% to about 60%) of the HCl solution over a third time period after the second time period. In various embodiments, the first, second and third time periods are independently in the range between about 10 minutes to about 90 minutes. In various embodiments, the first, second and third time periods are substantially equal within ±10% of each other.

### Example 1: Preparation of Crystalline (S)-1-(4,7-dihydro-5H-thieno[2,3-c]pyran-7-yl)-N-methylmethanamine hydrochloride (“(S)-Formula I HCl”) Form A

### Scheme 1: Preparation of 1-(4,7-dihydro-5H-thieno[2,3-c]pyran-7-yl)-N-methylmethanamine (Formula I) triflate

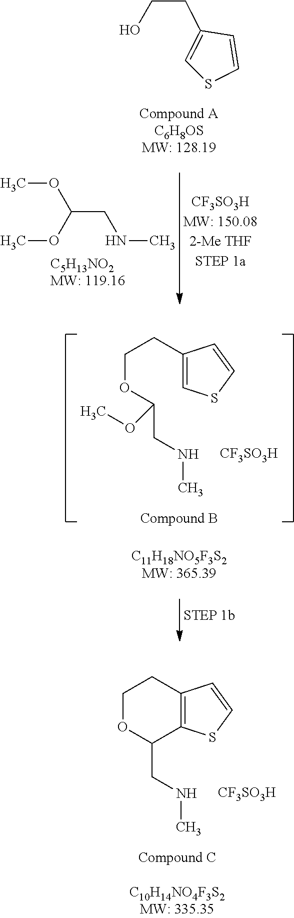

77 g of 3-thiopheneethanol (Compound A) was added to a solution of 69 g of N-methylaminoacetadehyde dimethyl acetal in 595 ml (508 g) of 2-methyl tetrahydrofuran (THF). After stirring for about 5 minutes, 99 g (58.2 ml) trifluoromethanesulfonic acid was added. The reaction was heated to reflux for about 1 hour (80±2° C.). The reaction was then distilled at about atmospheric pressure to remove the byproduct methanol and to reduce the reaction volume to a targeted volume of 460 ml over about 4-8 hours. The reaction was judged complete when about 1.0% or less (HPLC Peak Area % of peaks of interest, Compounds A, B and C) of compound 1B remained by a sample HPLC analysis.

If the amount of Compound B was greater than or equal to about 1%, an appropriate amount of 2-methyl THF was added and distillation continued to the target volume. If the target volume was reached before the completion of reaction (about 4 hours), 300 ml 2-methyl THF was added to the reaction for continuation of the distillation. After reaction completion, the reaction was cooled to about 40-50° C. and concentrated to a target volume of 325 ml under vacuum distillation. 218 g (325 ml) of Toluene was then added over about 15 minutes and the reaction slurry that formed was then stirred for about 1 hour at about 50±2° C., and then cooled to about 20±2° C. linearly over about 1 hour 45 minutes while being stirred. The slurry was filtered and the product cake was washed with a 2-methyl THF and toluene mixture (1:1 volume/volume). The wet-cake was dried under vacuum at about 40±5° C. to constant weight to yield racemic Formula I triflate (Compound C).

### Scheme 2: Preparation of (S)-(−)-Formula I (R)-mandelate

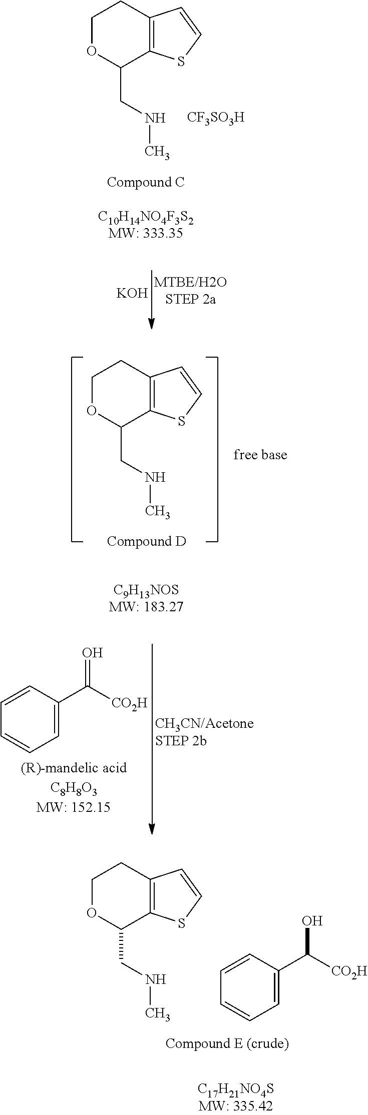

In some embodiments, di-p-toluoyl-D-tartaric acid (D-DTTA) was used as the resolving agent to produce a (S)-Formula I-D-DTTA salt and the present inventors discovered that use of D-DTTA provided for a kinetic based resolution. However, Scheme 2 of the present example provides for use of (R)-mandelic acid and the present inventors discovered that diasteromeric crystallization with (R)-mandelic acid is a thermodynamic based separation.

To a suspension of 555.3 g of Formula I triflate (Compound 1C) in 1668 ml methyl tert-butyl ether (MTBE) was added 1076 g of 1.77 N aqueous KOH. After stirring for about 10 minutes the pH was checked and if the pH was less than about 13, small amounts of 1.77 N KOH were added until the pH was 13 or greater. The aqueous and organic layers were allowed to settle and separate and separately collected. The MTBE (upper) organic phase layer was held for further processing. The aqueous (bottom) phase layer was extracted twice with MTBE (first with 835 ml and second with 150 ml), the organic (MBTE) layer being collected each time. The MTBE layers (organic layers) were combined, and washed with 20% aqueous NaCl solution (492.9 g) stirred and the phases allowed to settle for about 10 minutes. The aqueous layer was removed and the remaining MTBE organic layer was distilled at atmospheric pressure to reduce the reaction volume to a targeted level of 1.9 L. After completion, the process stream was cooled to about 45° C. and concentrated to a target volume of 890 ml under vacuum distillation while maintaining the temperature at about 35-45° C. The water content after vacuum distillation was found to be about 0.37% by weight. A filtration was then performed to remove insoluble materials using a wash of 204 ml MTBE, and the process stream (filtrate) was transferred to a clean reactor. 2512 mL of acetonitrile was added and a solvent switch was performed via vacuum distillation at about 35-45° C. to the targeted volume of 800 ml, and the reactor washed with 150 ml of acetonitrile and added to the process stream. Acetonitrile was then added, if needed, to the acetonitrile solution of Formula I free base (Compound D) to achieve about a 33 weight % of Compound D.

A solution of 250.3 g of (R)-mandelic acid in 1828 ml of acetone was warmed to 48±2° C. The Formula I free base solution in acetonitrile (917.7 g solution of 302.1 g of Compound D in acetonitrile) was then added at a rate maintaining the reaction temperature below about 51° C. After stirring at about 48±2° C. for about 10 minutes the process stream was cooled to about 45±2° C. and charged with 1.5 g of (S)-Formula I (R)-mandelate seed crystals. The process stream was stirred at about 45±2° C. for about 30 minutes and cooled linearly to about 21±2° C. over about 90 minutes. After holding at about 45±2° C. for about 30 minutes the process stream was cooled linearly to about 10±2° C. over about 45 minutes. The reaction slurry was then stirred for about 60 minutes at 10±2° C., filtered and the product cake was washed with acetone/CH₃CN mixture (2.3:1 weight/weight). The wet-cake was dried under vacuum at 40±2° C. to a constant weight to yield crude (S)-Formula I (R)-mandelate (Compound E).

### Scheme 3: Recrystallization of (S)-Formula I (R)-mandelate

Scheme 3 presents a process for the recrystallization of (S)-1-(4,7-dihydro-5H-thieno[2,3-c]pyran-7-yl)-N-methylmethanamine (R) mandelate, (“(S)-Formula I (R)-mandelate”). It is to be understood that various other recrystallization solvents can be used. Scheme 3 of the present example provides for use of acetone and the present inventors have discovered that acetone can provide a combination of sufficiently high yield and effective rejection of key impurities. In various embodiments, the amount of acetone was selected based on solubility of (S)-Formula I (R)-mandelate in acetone at reflux temperature, preferably the minimum amount of acetone required for dissolution of crude (S)-Formula I (R)-mandelate at reflux was used. In some embodiments, Scheme 3 is a seed-induced crystallization and is conducted with linear cooling from about 47±2° C. to about 21±2° C. over about 90 minutes followed by a hold for about 30 minutes at about 21±2° C., followed by linear cooling to about 10±2° C. over about 45 minutes and a hold at about 10+±2° C. preferably for a minimum of about 1 hour.

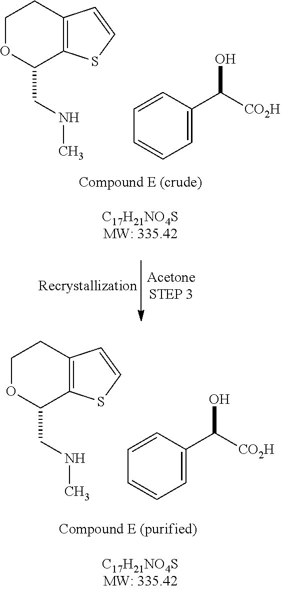

A slurry of crude (S)-Formula I (R)-mandelate (Compound E) from Scheme 2 (200.1 g) in 4205 ml of acetone was warmed to about 56° C. and stirred until a clear solution was obtained. After cooling the solution to about 47±2° C. over about 20 minutes (S)-Formula I (R)-mandelate seed crystals were added. The process stream was stirred at about 47±2° C. for about 30 minutes and cooled linearly to about 21±2° C. over about 90 minutes. After holding at about 21±2° C. for about 30 minutes the slurry was cooled linearly to about 10±2° C. over about 45 minutes and then stirred for about 1 hour at about 10±2° C., filtered, and the product cake was washed with acetone (twice with 401 ml each time). The wet-cake was dried under vacuum at about 40±2° C. to a constant weight to yield (S)-Formula I (R)-mandelate (purified Compound E).

### Scheme 4: Formation of Crystalline (S)-(−)-Formula I HCl Form A

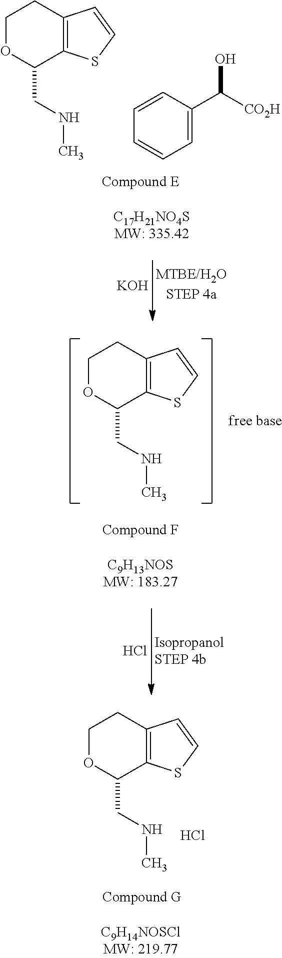

Scheme 4 of the present example provides a reactive crystallization of (S)-(−)-Formula I HOI as crystalline Form A. In addition to a new and unique crystalline form, the present inventors also believe that (S)-Formula I HCl is a new and inventive salt of (S)-Formula I. The present inventors have discovered that as (S)-Formula I HCl crystallizes it displays two distinct morphologies (polymorphs), the first a block like crystal (Form A) and the second a needle like crystal (Form B). Based on single crystal x-ray diffraction studies, described herein, Form A has a monoclinic crystal system while Form B has an orthorhombic crystal system. The present inventors have discovered that Form A is the stable form under the reaction conditions of the present example and have discovered how to avoid formation of Form B. In some embodiments, (S)-Formula I (R)-mandelate is first converted to the free base and HCl added to form a slurry.

To a suspension of (S)-Formula I (R)-mandelate salt (Compound E) from Scheme 3 (100 g) in 305 ml of MTBE, 172.5 ml of a 10% KOH aqueous solution was added. After stirring for about 10 minutes at about 20±2° C. the aqueous and organic layers were separated. The organic MTBE (upper) layer was saved for further processing. If the pH of the aqueous layer was less than 13, small amounts of the 19% KOH solution were added to raise the pH to 13. The aqueous (bottom) layer was back extracted twice with MTBE (first with 208 ml MTBE, second with 155 ml MTBE), the organic layer being saved for further processing each time. The saved organic layers were combined, and the combined organic layer was subjected to azeotropic distillation to remove water and distilled at atmospheric pressure to a target volume of 140 ml. The process stream was then filtered, to remove insoluble material (e.g. salt precipitated due to removal of water), and the filtrate transferred to a clean reactor. 775 ml of isopropanol was added (resulting in a total process stream volume of about 1030 ml) and a solvent switch was performed via vacuum distillation at less than 45° C. to provide a 10%-15% solution of (S)-Formula I in isopropanol.

In some embodiments, the amount of isopropanol added was selected so to adjust the freebase (Compound F) weight % concentration to 6-7%. The reaction mixture was cooled to 20±2° C., filtered, the filter washed with 78 ml isopropanol, and the filtrate transferred to a clean reactor. 201.6 g of a 6% HCl (w/w) solution in isopropanol was then added into the reactor over about 45 minutes at about 20±2° C. It is to be understood that in some embodiments, the target amount of HCl is about 10% excess relative to the freebase (Compound F) molar equivalence. The HCl was added as follows, the first 10% was added over about 15 minutes, the next 30% was added over about 15 minutes, and the remainder was then added over about 15 minutes. A retreat curve impeller at 160 rpm to 270 rpm in a 5 L scale reactor was used, with a process stream volume of about 740 ml, and produced reasonable-sized particles and particle distributions with no obvious agglomeration observed. The slurry formed was warmed up to about 40±2° C. linearly over about 20 minutes and held at about 40±2° C. for about 30 minutes. It was then cooled linearly to about 20±2° C. over about 20 minutes. After stirring at about 20±2° C. for about 30 minutes the slurry was filtered and the product cake was washed with isopropanol (first with 86 ml, second with 92 ml). The cake was dried under vacuum at 40±2° C. to a constant weight to yield (S)-(−)-Formula I HCl (Compound G).

In Step 4b of Scheme 4, slow addition, that results in low supersaturation generation rate, favors the formation of desired block (S)-(−)-Formula I HCl Form A crystals while decreasing the generation the undesired needles (Form B). Higher temperature also favored the formation of the block like Form A crystals over Form B.

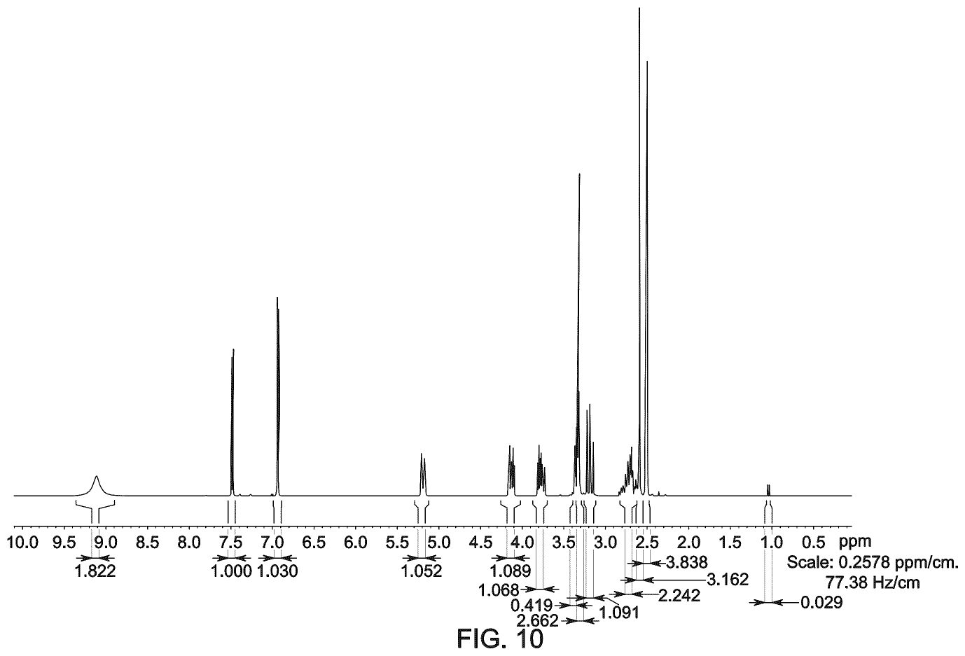

An ¹H NMR spectrum of the (S)-(−)-Formula I HCl (Compound G) obtained in Example 1 is illustrated in FIG. 10, having the following characteristics: ¹H NMR (300 MHz, DMSO-d₆); δ (ppm): 2.53 (s, 3H, —CH₃); 2.5-2.8 (m, 2H, —CH₂—); 3.15-3.37 (2dd, 2H, CH₂—NH); 3.77 and 4.13 (2ddd, 2H, CH₂—O); 5.19 (dd, 1H, O—CH—C═); 6.95 (d, J=5 Hz, 1H, HC═); 7.49 (dd, J=5 Hz, 1H, HC═); 9.12 (br, 2H, NH₂⁺).

### Example 2: Particle Size Distribution Control of (S)-Formula I HCl Form A Crystals

A series of experiments was conducted on various aspects of the reactive-recrystallization (e.g. Scheme 4 in Example 1) to develop methods and provide various particle size distribution of (S)-(−)-Formula I HCl Form A crystals. Reaction conditions were substantially similar to those set for in Example 1 with respect to Scheme 4 except as modified as described in this Example 2.

The PSD data of this Example 2 was obtained using laser diffraction particle sizing of the sample dispersed in a solvent. The data of FIG. 7A, FIG. 7B, FIG. 8A, FIG. 8B and FIG. 8C was obtained using a Malvern Mastersizer 2000 analyzer, and the data of FIG. 9A was obtained using a Horiba LA-920 laser diffraction particle size analyzer. All particle sizes and D(4,3), D10, D50, D90, etc. values are in micrometers (μm), and all distributions are for volume % as a function of particle size.

The (S)-Formula I HCl sample was dispersed in a solution of Span®-85 (sorbitan trioleate) and hexanes. In this Example, the dispersant solution was 2 g of Span®-85 in 1 liter of hexanes, to make a 0.2% (w/v) Span®-85 in hexanes solution. All samples were gently sieved through a #30 mesh screen prior to addition to the dispersant solution.

The suspension solution for analysis was prepared by addition of approximately 5 mL of the 0.2% Span®-85 in hexanes dispersant solution to 1.5 to 3 grams of the sieved (S)-Formula I HCl sample, and the solution gently swirled until all of the solids were wetted. Then 35 mL of the 0.2% Span®-85 in hexanes dispersant solution was added and the solution mixed for at least 1 minute prior to measurement with an impeller set to 500 rpm to make the suspension solution. The actual amount of (S)-Formula I HCl sample, to which the dispersant solution is added, was determined experimentally and adjusted such that 2 to 3 mL of the resultant suspension solution will result in a laser obscuration between 10% and 20% as measured by the instrument used.

Prior to measurement, the instrument was aligned and background measured, and 2-3 mL of the suspension solution transferred to the sample cell of the instrument for measurement.

The data of FIG. 7A, FIG. 7B, FIG. 8A, FIG. 8B and FIG. 8C was obtained using a Malvern Mastersizer 2000 analyzer, and Table 6 provides further details on the instrument settings of the Malvern Mastersizer 2000 analyzer used in this Example. Corresponding and similar setting were used on the Horiba LA-920 laser diffraction particle size analyzer used to acquire the data of FIG. 9A.

TABLE 6
Malvern Mastersizer 2000 Analyzer Instrument Settings

| Parameter | Setting | |
|---|---|---|
| Stirrer/Pump Speed | 1750 | rpm |
| Ultrasound | 0 | |
| Sample Refractive Index | 1.5 | (red and blue light) |
| Sample Absorption | 0 | (red and blue light) |
| Dispersant name | 0.2% Span 85 in hexanes | |
| Dispersant Refractive Index | 1.38 | |
| Model | General Purpose - normal sensitivity | |
| Sample measurement time | 30 | seconds |
| Sample measurement snaps | 30000 | |
| Background measurement time | 30 | seconds |

| | |
|---|---|
| Background measurement snaps | 30000 |
| Number of measurement cycles | 1 |

### Modulation by Supersaturation Generation Rate

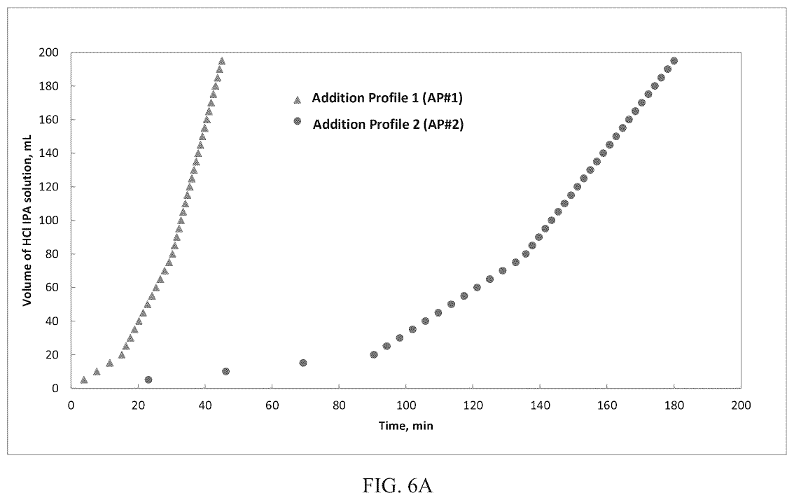

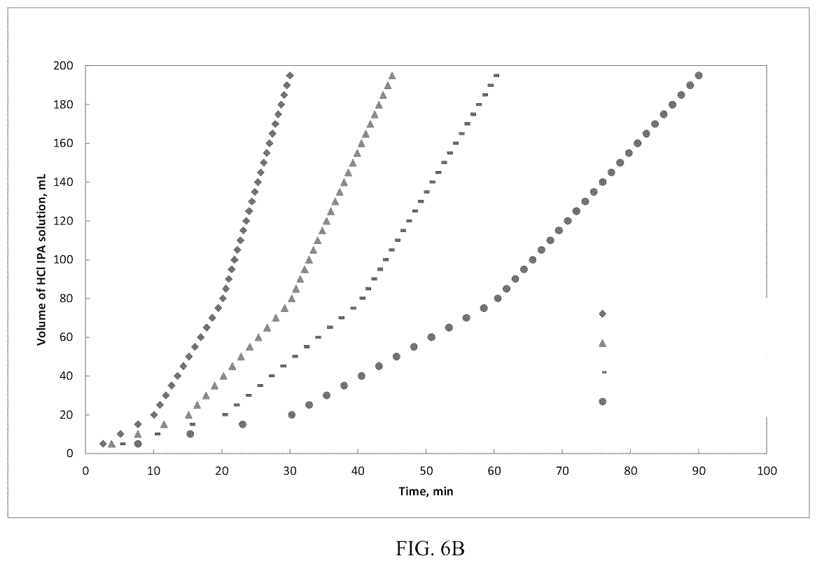

The (S)-(−)-Formula I freebase containing solution (e.g. solution of Compound F in Scheme 4) was reactively-recrystallized as a crystalline form of the (S)-(−)-Formula I HCl salt by addition of an HCl in isopropanol (IPA) to form a super saturated (S)-(−)-Formula I HCl from which crystallization occurred. FIG. 6A and FIG. 6B present various 6% HCl in LPA addition profiles, which are also summarized in Table 7. Measured resultant PSD for the addition profiles of FIG. 6A and FIG. 6B are presented respectively in FIG. 7A and FIG. 7B. Table 8 provides various PSD parameters of the PSD data presented in FIG. 7A and FIG. 7B.

It was discovered that a logarithmic-like addition of the reagent (HCl in IPA) responsible for supersaturation favored formation of Form A crystals and that a slower addition rate resulted in a larger median particle size and a lower span to the PSD.

TABLE 7
HCL IPA Solution Dosing Profiles

| Profile | HCl IPA solution addition |
|---|---|
| Addition | (i) first 10% added over approximately 15 minutes |
| Profile 1 | (ii) next 30% added over approximately 15 minutes |
| (AP#1) | (iii) remainder added over approximately 15 minutes |
| Addition | (i) first 10% added over approximately 90 minutes |
| Profile 2 | (ii) next 30% added over approximately 45 minutes |
| (AP#2) | (iii) remainder added over approximately 45 minutes |
| Addition | (i) first 10% added over approximately 10 minutes |
| Profile 4 | (ii) next 30% added over approximately 10 minutes |
| (AP#3) | (iii) remainder added over approximately 10 minutes |
| Addition | (i) first 10% added over approximately 15 minutes |
| Profile 4 | (ii) next 30% added over approximately 15 minutes |
| (AP#4) | (iii) remainder added over approximately 15 minutes |
| Addition | (i) first 10% added over approximately 20 minutes |
| Profile 5 | (ii) next 30% added over approximately 20 minutes |
| (AP#5) | (iii) remainder added over approximately 20 minutes |
| Addition | (i) first 10% added over approximately 30 minutes |
| Profile 6 | (ii) next 30% added over approximately 30 minutes |
| (AP#6) | (iii) remainder added over approximately 30 minutes |

TABLE 8
Particle Size Distribution Parameters for Dosing Profiles

| | D(4, 3) | D10 | D50 | D90 |
|---|---|---|---|---|
| Profile | (μm) | (μm) | (μm) | (μm) |
| Addition Profile 1 | 109.50 | 74.66 | 105.41 | 149.78 |
| Addition Profile 2 | 170.79 | 114.16 | 164.31 | 236.27 |
| Addition Profile 3 | 149.43 | 55.23 | 131.21 | 273.25 |
| Addition Profile 4 | 185.80 | 79.73 | 167.51 | 323.92 |
| Addition Profile 5 | 209.45 | 103.82 | 199.11 | 335.44 |
| Addition Profile 6 | 222.06 | 129.01 | 209.38 | 334.41 |

### Modulation by Temperature

The (S)-(−)-Formula I freebase containing solution (e.g. solution of Compound F in Scheme 4) was reactively-recrystallized as a crystalline form of the (S)-(−)-Formula I HCl salt by addition of an HCl in isopropanol (IPA) at two different temperatures, 25° C. and 40° C. Table 9 provides various PSD parameters of the measured PSD data at these two temperatures.

It was discovered that increasing temperature increased the median and mean particle size of the Form A crystals of (S)-(−)-Formula I HCl but increased temperature also increased the span of the PSD.

TABLE 9
Particle Size Distribution Parameters for Various Temperatures

| | D(4, 3) | D10 | D50 | D90 |
|---|---|---|---|---|
| Temperature | (μm) | (μm) | (μm) | (μm) |
| 40° C. | 180 | 86 | 164 | 302 |
| 25° C. | 109 | 65 | 102 | 167 |

### Modulation by Freebase Concentration

The (S)-(−)-Formula I freebase containing solution (e.g. solution of Compound F in Scheme 4) was reactively-recrystallized as a crystalline form of the (S)-(−)-Formula I HCl salt by addition of an HCl in isopropanol (IPA) from three different starting concentrations of (S)-(−)-Formula I freebase, 10.8%, 13.0% and 15.2%. Table 10 provides various PSD parameters of the measured PSD data presented in FIG. 8A, FIG. 8B, and FIG. 8C; where FIG. 8A presents PSD data for a 15.2% (S)-(−)-Formula I freebase concentration, FIG. 8B presents PSD data for a 13.0% (S)-(−)-Formula I freebase concentration, and FIG. 8C presents PSD data for a 10.8% (S)-(−)-Formula I freebase concentration.

It was discovered that increasing starting (S)-(−)-Formula I freebase concentration decreased both the median particle size and the PSD span and that decreasing the starting (S)-(−)-Formula I freebase concentration increased the both the median particle size and the PSD span.

TABLE 10
Particle Size Distribution Parameters for
Various Freebase Concentrations

| Freebase Concentration | D(4, 3) | D10 | D50 | D90 |
|---|---|---|---|---|
| (weight %) | (μm) | (μm) | (μm) | (μm) |
| 15.2% | 104 | 66 | 99 | 148 |
| 13.0% | 109 | 65 | 102 | 167 |
| 10.8% | 134 | 55 | 124 | 228 |

### Modulation by Water Content

The (S)-(−)-Formula I freebase containing solution (e.g. solution of Compound F in Scheme 4) was reactively-recrystallized as a crystalline form of the (S)-(−)-Formula I HCl salt by addition of an HCl in isopropanol (IPA) from solutions of (S)-(−)-Formula I freebase with different water content (i.e. pre-nucleation water content), ranging from 2%-5.5%. Table 11 provides various PSD parameters of the measured PSD data for the indicated water content.

It was discovered that increased water content generally resulted in increased median particle size but decreased PSD span.

TABLE 11
Particle Size Distribution Parameters for Various Water Contents

| Water Content | D(4, 3) | D10 | D50 | D90 |
|---|---|---|---|---|
| (before nucleation) | (μm) | (μm) | (μm) | (μm) |
| 2% | 189.0 | 120.8 | 179.6 | 268.7 |
| 2.5% | 154.4 | 77.5 | 140.4 | 249.3 |
| 3% | 160.6 | 97.2 | 148.4 | 236.7 |
| 3.5% | 158.4 | 100.3 | 150.0 | 225.5 |
| 4% | 201.1 | 116.8 | 192.3 | 294.6 |
| 5% | 216.8 | 115.1 | 204.9 | 332.6 |
| 5% | 191.9 | 105.9 | 173.9 | 297.9 |
| 5.5% | 220.7 | 141.4 | 211.3 | 309.0 |

### Modulation by Reaction Process

The reactive-recrystallization was carried out by two different process, (i) Process 1 employing a Plug Flow Reactor (PFR) process with ultrasound applied to the reaction mixture during nucleation (e.g. during Step 4b of Scheme 4); and (ii) Process 2 a multi-stage mixed suspension and mixed product removal (MSMPR) process.

The chemistry, e.g., chemicals, concentrations, and stoichiometry, used in the reactive-recrystallization under Process 1 and Process 2, were substantially similar to that of Example 1 where Process 1 and Process 2 starting with the (S)-(−)-Formula I free base solution (Compound F) of Scheme 4 in Example 1 of various concentrations.

Reactive-recrystallization under Process 1 was conducted as follows. The (S)-(−)-Formula I free base solution and the HC/IPA solution were pumped, using peristaltic pumps, as separate feed streams into a tubing crystallizer through a Tee mixer, at a controlled temperature (e.g., 40° C.) and residence time, to perform Step 4b of Scheme 4. The crystallization occurred as the process stream flowed through the tubing after contact at the Tee. A N₂ injection system was integrated into both feed streams to enable periodic introduction of gas. The output solution, post mixer Tee, was passed through a tubular coil (⅛″ PFA tubing) of predetermined length depending on the desired residence time. For a residence time of about 2.5 minutes a coil length of 3.5 m was used, and for a residence time of about 5 minutes a coil length of 7 m was used. The temperature control for the coil was achieved using a water bath in which the Tee, approximately 10 cm of each of the input stream tubes, and the coil were immersed, and sonication was achieved by sonication of the water bath during process flow.

Reactive-recrystallization under Process 2 was conducted as follows. The multi-sage MSMPR process employed three stages with process streams continually pumping starting materials into a first reaction vessel (first stage crystallizer), continually pumping products out of the first reaction vessel into a second reaction vessel (second stage crystallizer), continually pumping products out of the second reaction vessel into a third reaction vessel (third stage crystallizer) and continually pumping products out of the third reaction vessel to a product receiving vessel. The operation volume and reaction conditions were kept steady state during the process and each reaction vessel was stirred.

A starting (S)-(−)-Formula I free base isopropanol solution and 13% of the HCl isopropanol solution were pumped into the first stage with set flow rates to control the residence time and the ratio of (S)-(−)-Formula I free base to HCl for each stage. The suspension from the first stage crystallizer was transferred to the second stage crystallizer and 37% of the HCl isopropanol solution was pumped to the second stage crystallizer. The suspension from the second stage crystallizer was transferred to the third stage crystallizer and the reminder (50%) of the HCl isopropanol solution was pumped to the third stage crystallizer. Pumping was performed with peristaltic pumps. The various flow and other conditions for each stage are summarized in Table 12.

TABLE 12
MSMPR Stage Conditions and Parameters

| STAGE 1 | | |
|---|---|---|
| | Average volume (mL) | 65.00 |
| | Tau 1 (min) | 10.00 |
| | Overall flow rate in Stage 1 (mL/min) | 6.50 |
| | Slug volume (mL) | 10.00 |
| | Slug interval (min) | 1.54 |
| | Feed flow rate (mL/Min) | 6.12 |
| | HCL in IPA flow rate (mL/min) | 0.38 |
| | Operating temperature (° C.) | 40 |
| | Agitation rate, reaction vessel stirring, (rpm) | 300 |
| STAGE 1 | | |
| | Average volume (mL) | 75.8 |
| | Tau 1 (min) | 10.00 |
| | Overall flow rate in Stage 1 (mL/min) | 7.58 |
| | Slug volume (mL) | 11.66 |
| | Slug interval (min) | 1.54 |
| | HCL in IPA flow rate (mL/min) | 1.08 |
| | Operating temperature (° C.) | 40 |
| | Agitation rate, reaction vessel stirring, (rpm) | 300 |
| STAGE 1 | | |
| | Average volume (mL) | 90.3 |
| | Tau 1 (min) | 10.00 |
| | Overall flow rate in Stage 1 (mL/min) | 9.03 |
| | Slug volume (mL) | 13.89 |
| | Slug interval (min) | 1.54 |
| | HCL in IPA flow rate (mL/min) | 1.45 |
| | Operating temperature (° C.) | 40 |
| | Agitation rate, reaction vessel stirring, (rpm) | 300 |

Table 13 provides various PSD parameters of the measured PSD data presented in FIG. 9A; and FIG. 9B and FIG. 9C present SEM images of crystalline (S)-(−)-Formula I HCl Form A obtained by Process 2 (FIG. 9B) and Process 1 (FIG. 9C).

It was discovered that sonication during the step of supersaturation provided a PSD with a small median particle size and an acceptable PSD span. On addition, it was discovered that sonication during the step of supersaturation favors primary nucleation of the block-like crystal form (Form A) of (S)-(−)-Formula I HCl, and facilitates avoiding the needle form (Form B).

TABLE 13
Particle Size Distribution Parameters for
Various Reaction Processes

| | D(4, 3) | D10 | D50 | D90 |
|---|---|---|---|---|
| Reaction Process | (μm) | (μm) | (μm) | (μm) |
| Process 1 (PRF with ultra-sonication) | 21.9 | 11.4 | 20.3 | 34.8 |
| Process 2 (multi-stage MSMPR) | 210.6 | 77.0 | 190.2 | 377.1 |

In various embodiments, crystalline forms of the present inventions have several advantageous physical properties. For example, crystalline (S)-Formula I HCl Form A is substantially non-hygroscopic, in some embodiments exhibiting less than about a 0.2%, and preferably less than about 0.1%, maximum mass change in water sorption isotherms, at 25° C. scanned over 0 to 90% relative humidity, as measured by dynamic vapor sorption (DVS) (see, for example, FIG. 5).

It is to be understood that some embodiments provide crystalline (S)-Formula I HCl Form A, in high chiral purity and high chemical purity.

In some embodiments the present inventions provide substantially enantiomerically pure crystalline forms of (S)-Formula I HCl Form A. For example, in some embodiments, the present inventions provide crystalline forms of Formula I HCl that contain greater than about 90% (S)-Formula I HCl and less than about 10% of (R)-Formula I HCl, greater than about 95% (S)-Formula I HCl and less than about 5% of (R)-Formula I HCl, greater than about 97% (S)-Formula I HCl and less than about 3% of (R)-Formula I HCl, greater than about 99% (S)-Formula I HCl and less than about 1% of (R)-Formula I HCl, greater than about 99.5% (S)-Formula I HCl and less than about 0.5% of (R)-Formula I HCl, greater than about 99.7% (S)-Formula I HCl and less than about 0.3% of (R)-Formula I HCl, or greater than about 99.9% (S)-Formula I HCl and less than about 0.1% of (R)-Formula I HCl.

In some embodiments, provided are substantially chemically pure crystalline forms of (S)-Formula I HCl Form A. In some embodiments, provided is crystalline (S)-Formula I HCl Form A that has a greater than about 80% chemical purity, greater than about 90% chemical purity, greater than about 95% chemical purity, greater than about 97% chemical purity, greater than about 99% chemical purity, greater than about 99.5% chemical purity, greater than about 99.7% chemical purity, or greater than about 99.9% chemical purity. In some embodiments, provided is crystalline (S)-Formula I HCl Form A that has less than about 8000 ppm residual solvents, less than about 6000 ppm residual solvents, less than about 4000 ppm residual solvents, less than about 2000 ppm residual solvents, less than about 1000 ppm residual solvents, less than about 800 ppm residual solvents, or less than about 500 ppm residual solvents.

### Compositions

In certain embodiments, the present disclosure provides a pharmaceutical composition comprising a compound of the present disclosure (e.g. a compound of Formula I or isomer thereof) or a pharmaceutically acceptable salt thereof, and a pharmaceutically acceptable excipient as defined herein. In certain embodiments, the present disclosure provides a pharmaceutical composition comprising a compound of Formula I, or a pharmaceutically acceptable salt thereof, and one or more pharmaceutically acceptable excipient(s).

Pharmaceutical compositions comprising the compounds disclosed herein, or a pharmaceutically acceptable salt thereof, may be prepared with one or more pharmaceutically acceptable excipients selected according to ordinary practice.

In some embodiments, the compositions are formulated with one or more pharmaceutically acceptable excipients in accordance with known and established practice. Thus, in various embodiments the composition are formulated as, for example, a liquid, powder, elixir, injectable solution, or suspension. Formulations for oral use are preferred and may be provided, for instance, as tablets, caplets, or capsules, wherein the pharmacologically active ingredients are mixed with an inert solid diluent. Tablets may also include granulating and disintegrating agents, and may be coated or uncoated. Formulations for topical use may be provided, for example as topical solutions, lotions, creams, ointments, gels, foams, patches, powders, solids, sponges, tapes, vapors, pastes or tinctures.

Pharmaceutical compositions disclosed herein include those suitable for various routes of administration, including enteral, parenteral, and/or topical administration. Pharmaceutical compositions disclosed herein may be administered orally, parenterally, by inhalation, topically, rectally, nasally, buccally, sublingually, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the compositions of the present inventions may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, such as, for example, as a solution in 1,3-butanediol. Acceptable vehicles and solvents that may be employed include, but are not limited to, water, Ringer's solution and isotonic sodium chloride solution. In addition, sterile, fixed oils are conventionally employed as a solvent or suspending medium. Pharmaceutically acceptable compositions of this invention may be orally administered in any orally acceptable dosage form including capsules, tablets, aqueous suspensions or solutions.

The compositions may be presented in unit dosage form and may be prepared by any of the methods well known in the art of pharmacy. Such methods include the step of bringing into association the active ingredient (e.g., a compound of the present disclosure or a pharmaceutical salt thereof) with one or more pharmaceutically acceptable excipients. The compositions may be prepared by uniformly and intimately bringing into association the active ingredient with liquid excipients or finely divided solid excipients or both, and then, if necessary, shaping the product. Techniques and formulations generally are found in Remington: The Science and Practice of Pharmacy, 21.sup.st Edition, Lippincott Williams and Wilkins, Philadelphia, Pa., 2006. In some embodiments the composition are formulated as a liquid, powder, elixir, injectable solution, or suspension. In some embodiments, provided are formulations for oral use as tablets, caplets, or capsules, wherein the pharmacologically active ingredients are mixed with an inert solid diluent. Tablets may also include granulating and disintegrating agents, and may be coated or uncoated. Formulations for topical use may be provided, for example as topical solutions, lotions, creams, ointments, gels, foams, patches, powders, solids, sponges, tapes, vapors, pastes or tinctures.

Compositions described herein that are suitable for oral administration may be presented as discrete units (a unit dosage form) including but not limited to troches, lozenges, aqueous or oil suspensions, dispersible powder or granules, emulsions, hard or soft capsules, cachets, syrups, elixirs, or tablets each containing a predetermined amount of the active ingredient. In certain embodiments, the pharmaceutical composition is a solid oral dosage. In certain embodiments, the pharmaceutical composition is a tablet.

Compositions described herein need not be provided in a single unit dosage form, e.g. a single tablet, capsule, etc. In some embodiments, the pharmaceutical composition is provided in unit dosage forms such that administration of two of the unit dosage forms result in administration of the desired amount of the compound of Formula I, or a pharmaceutically acceptable salt thereof.

Pharmaceutical compositions disclosed herein comprise one or more compounds disclosed herein, or a pharmaceutically acceptable salt thereof, together with a pharmaceutically acceptable excipient and optionally other therapeutic agents. Pharmaceutical compositions containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more excipients, for example, sweetening agents, flavoring agents, coloring agents and preserving agents, in order to provide a palatable preparation.

Tablets containing the active ingredient in admixture with non-toxic pharmaceutically acceptable excipients which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

The amount of active ingredient that may be combined with the inactive ingredients to produce a dosage form may vary depending upon the intended treatment subject and the particular mode of administration. For example, in some embodiments, a dosage form for oral administration to humans may contain approximately 0.1 to 1000 mg of active material formulated with an appropriate and convenient amount of a pharmaceutically acceptable excipient. In certain embodiments, the pharmaceutically acceptable excipient varies from about 5 to about 95% of the total compositions (weight:weight).

In certain embodiments, a composition comprising a compound of the present disclosure, for example, the compound of Formula I, or a pharmaceutically acceptable salt thereof, in one variation does not contain an agent that affects the rate at which the active ingredient is metabolized. Thus, it is understood that compositions comprising a compound of the present disclosure in one aspect do not comprise an agent that would affect (e.g., slow, hinder or retard) the metabolism of a compound of the present disclosure or any other active ingredient administered separately, sequentially or simultaneously with a compound of the present disclosure. It is also understood that any of the methods, kits, articles of manufacture and the like detailed herein in one aspect do not comprise an agent that would affect (e.g., slow, hinder or retard) the metabolism of a compound of the present disclosure or any other active ingredient administered separately, sequentially or simultaneously with a compound of the present disclosure.

Compositions comprising the compounds disclosed herein, or pharmaceutically acceptable salts thereof, can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566. For example, hydropropylmethyl cellulose, other polymer matrices, gels, permeable membranes, osmotic systems, multilayer coatings, microparticles, liposomes, microspheres, or a combination thereof provide the desired release profile in varying proportions. Suitable controlled-release formulations known to those of ordinary skill in the art can be readily selected for use with compounds disclosed herein, or pharmaceutically acceptable salts thereof.

Compositions comprising the compounds disclosed herein, or pharmaceutically acceptable salts thereof, for parenteral administration include aqueous and non-aqueous sterile injection solutions, which may contain anti-oxidants, buffers, bacteriostats and solutes that render the formulation isotonic with the blood of the intended recipient. Parenteral administration also include aqueous and non-aqueous sterile suspensions, which may include suspending agents and thickening agents. Compositions comprising the compounds disclosed herein, or pharmaceutically acceptable salts thereof, may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

In some embodiments, the composition comprises about 1 mg to 1,000 mg of the compound of Formula I, or a pharmaceutically acceptable salt thereof. In some embodiments, the composition comprises about 1 mg to 150 mg of the compound of Formula I, or a pharmaceutically acceptable salt thereof. In some embodiments, the composition comprises about 30 mg to 120 mg of the compound of Formula I, or a pharmaceutically acceptable salt thereof. In some embodiments, the composition comprises about 30 mg to 90 mg of the compound of Formula I, or a pharmaceutically acceptable salt thereof.

In some embodiments, provided are pharmaceutical compositions comprising a compound of Formula I, or a pharmaceutically acceptable salt thereof, and/or crystalline forms thereof, and one or more pharmaceutically acceptable excipients. In some embodiments, provided are pharmaceutical compositions comprising about 1 mg to 1,000 mg of a compound of Formula I, or a pharmaceutically acceptable salt thereof, and/or crystalline forms thereof, and one or more pharmaceutically acceptable excipients. In some embodiments, provided are pharmaceutical compositions comprising about 1 mg to 150 mg of a compound of Formula I, or a pharmaceutically acceptable salt thereof, and/or crystalline forms thereof, and one or more pharmaceutically acceptable excipients. In some embodiments, provided are pharmaceutical compositions comprising about 30 mg to 120 mg of a compound of Formula I, or a pharmaceutically acceptable salt thereof, and/or crystalline forms thereof, and one or more pharmaceutically acceptable excipients. In some embodiments, provided are pharmaceutical compositions comprising about 30 mg to 90 mg of a compound of Formula I, or a pharmaceutically acceptable salt thereof, and/or crystalline forms thereof, and one or more pharmaceutically acceptable excipients.

In some embodiments, provided are pharmaceutical compositions comprising a (S)-Formula I, or a pharmaceutically acceptable salt thereof, and/or crystalline forms thereof, and one or more pharmaceutically acceptable excipients. In some embodiments, provided are pharmaceutical compositions comprising about 1 mg to 1,000 mg of (S)-Formula I, or a pharmaceutically acceptable salt thereof, and/or crystalline forms thereof, and one or more pharmaceutically acceptable excipients. In some embodiments, provided are pharmaceutical compositions comprising about 1 mg to 150 mg of (S)-Formula I, or a pharmaceutically acceptable salt thereof, and/or crystalline forms thereof, and one or more pharmaceutically acceptable excipients. In some embodiments, provided are pharmaceutical compositions comprising about 30 mg to 120 mg of (S)-Formula I, or a pharmaceutically acceptable salt thereof, and/or crystalline forms thereof, and one or more pharmaceutically acceptable excipients. In some embodiments, provided are pharmaceutical compositions comprising about 30 mg to 90 mg of (S)-Formula I, or a pharmaceutically acceptable salt thereof, and/or crystalline forms thereof, and one or more pharmaceutically acceptable excipients.

In some embodiments, provided are pharmaceutical compositions comprising a (R)-Formula I, or a pharmaceutically acceptable salt thereof, and/or crystalline forms thereof, and one or more pharmaceutically acceptable excipients. In some embodiments, provided are pharmaceutical compositions comprising about 1 mg to 1,000 mg of (R)-Formula I, or a pharmaceutically acceptable salt thereof, and/or crystalline forms thereof, and one or more pharmaceutically acceptable excipients. In some embodiments, provided are pharmaceutical compositions comprising about 1 mg to 150 mg of (R)-Formula I, or a pharmaceutically acceptable salt thereof, and/or crystalline forms thereof, and one or more pharmaceutically acceptable excipients. In some embodiments, provided are pharmaceutical compositions comprising about 30 mg to 120 mg of (R)-Formula I, or a pharmaceutically acceptable salt thereof, and/or crystalline forms thereof, and one or more pharmaceutically acceptable excipients. In some embodiments, provided are pharmaceutical compositions comprising about 30 mg to 90 mg of (R)-Formula I, or a pharmaceutically acceptable salt thereof, and/or crystalline forms thereof, and one or more pharmaceutically acceptable excipients.

In some embodiments, provided are pharmaceutical compositions comprising (S)-Formula I HCl, and/or crystalline forms thereof, and one or more pharmaceutically acceptable excipients.

In some embodiments, provided are pharmaceutical compositions comprising about 2.4% w/w to about 60% w/w of the compound of Formula I, or a pharmaceutically acceptable salt thereof. In some embodiments, provided are pharmaceutical compositions comprising about 2.4% w/w to about 60% w/w of the compound of Formula I HCl. In some embodiments, provided are pharmaceutical compositions comprising about 10% w/w to about 40% w/w of the compound of Formula I HCl.

In some embodiments, provided are pharmaceutical compositions comprising about 2.4% w/w to about 60% w/w of (S)-Formula I, or a pharmaceutically acceptable salt thereof. In some embodiments, provided are pharmaceutical compositions comprising about 2.4% w/w to about 60% w/w of (S)-Formula I HCl. In some embodiments, provided are pharmaceutical compositions comprising about 10% w/w to about 40% w/w of (S)-Formula I HCl.

In some embodiments, provided are pharmaceutical compositions comprising about 2.4% w/w to about 60% w/w of (R)-Formula I, or a pharmaceutically acceptable salt thereof. In some embodiments, provided are pharmaceutical compositions comprising about 2.4% w/w to about 60% w/w of (R)-Formula I HCl. In some embodiments, provided are pharmaceutical compositions comprising about 10% w/w to about 40% w/w of (R)-Formula I HCl.

In some embodiments, provided are pharmaceutical compositions comprising a compound of Formula I, or a pharmaceutically acceptable salt thereof, and one or more excipients selected from the group consisting of fillers, disintegrants, and lubricants. In some embodiments, provided are pharmaceutical compositions comprising a compound of Formula I, or a pharmaceutically acceptable salt thereof, and one or more excipients selected from the group consisting of fillers, disintegrants, and lubricants, wherein the filler is microcrystalline cellulose and mannitol, the disintegrant is sodium starch glycolate, and the lubricant is magnesium stearate. In some embodiments, provided are pharmaceutical compositions comprising a compound of Formula I, or a pharmaceutically acceptable salt thereof, and one or more excipients selected from the group consisting of fillers, disintegrants, and lubricants, wherein the filler is microcrystalline cellulose, the disintegrant is sodium starch glycolate, and the lubricant is magnesium stearate.

In some embodiments, provided are pharmaceutical compositions comprising 20 mg to 150 mg compound of Formula I, or a pharmaceutically acceptable salt thereof, 50 mg to 100 mg of one filler, 150 to 250 mg of a second filler, 5 mg to 15 mg disintegrant, and 0.5 mg to 3.0 mg lubricant.

In some embodiments, provided are tablets comprising:
(a) a core comprising: (i) about 2.4% w/w to about 60% w/w of a compound of Formula I HCl; (ii) microcrystalline cellulose and mannitol as filler; (iii) sodium starch glycolate as disintegrant; (iv) magnesium stearate as lubricant; and optionally (v) colloidal silicon dioxide (if needed) as glidant; and
(b) a coating comprising: (i) a (hydroxypropyl) methyl cellulose (HPMC)/hydroxypropylcellulose (HPC) matrix as a polymer coating system; and optionally one or more of: (ii) titanium dioxide as opacifier and colorant, (iii) carnauba wax as polishing agent, and (iv) and other colorants to provide various tablet colors for, e.g., market need.

In some embodiments, the concentration of each ingredient is selected based on powder flowability, tabletability and tablet stability after storage at accelerated and long-term conditions.

### Example 3: Examples of Pharmaceutical Composition

Non-limiting exemplary tablets comprising (S)-Formula I HCl were manufactured. Tablets comprising 25 mg of (S)-Formula I HCl were manufactured by dry, direct compression. Components of the 25 mg tablet are summarized in Table 14.

Tablets comprising 50 mg, 75 mg, and 100 mg of (S)-Formula I HCl were manufactured by dry granulation. Components of the 50 mg, 75 mg, and 100 mg tablets are summarized in Table 15.

For the dosage strength of 25 mg based on the amount of free base, (i.e. (S)-Formula I) (S)-Formula I HCl, microcrystalline cellulose, mannitol, and sodium starch glycolate were sieved individually through a #30 mesh screen and charged into a low shear blender. The mixture was blended for up to 500 revolutions. Magnesium stearate was sieved though a #60 mesh screen, charged into the blender and the mixture blended for an additional 75 revolutions. The blend was then compressed into tablets with a target tablet weight of 300 mg. The tablets were then coated with Opadry 20A120006 Yellow, Opadry 20A18407 White or Opadry 20A110008 Green (hydroxypropylmethyl cellulose/hydroxypropyl cellulose), and carnauba wax was applied onto the tablets after drying.

For the dosage strengths greater than 25 mg based on the amount of free base, (i.e. (S)-Formula I), an intra-granular blend included (S)-Formula I HCl, microcrystalline cellulose, and sodium starch glycolate, which were sieved individually through a #30 mesh screen and charged into a low shear blender. The mixture was blended for up to 500 revolutions. Magnesium stearate was sieved though a #60 mesh screen, charged into the blender and the mixture blended for additional 75 revolutions. The intra-granular blend was then dry granulated into ribbons, and milled into granules. After dry granulation, the granules and the extra-granular excipients were blended before compression. The final blend included (S)-Formula I HCl granule, microcrystalline cellulose, mannitol, sodium starch glycolate, colloidal silicon dioxide (for 75 and 100 mg only) and magnesium stearate. Microcrystalline cellulose, mannitol, sodium starch glycolate and colloidal silicon dioxide were sieved individually or co-sieved with microcrystalline cellulose (for colloidal silicon dioxide only) through a #30 mesh screen and charged into a low shear blender with (S)-Formula I hydrochloride granule for blending. The mixture was blended for 250 revolutions. Extra-granular magnesium stearate was sieved through a #60 mesh screen and charged into the blender. The mixture was then blended for 75 revolutions and then compressed into tablets with target tablet weight of 300 mg. The tablets were then coated with Opadry 20A120006 Yellow, Opadry 20A18407 White or Opadry 20A110008 Green (hydroxypropylmethyl cellulose/hydroxypropyl cellulose), and carnauba wax was applied onto the tablets after drying.

TABLE 14
Example Pharmaceutical Composition (Tablet)
Comprising (S)-Formula I HCl (Dose Strength 25 mg)

| | |
|---|---|
| | Composition |
| Ingredient | (mg/tablet) |

Core Tablet

| | |
|---|---|
| (S)-Formula I HCl | 30.00 |
| Microcrystalline Cellulose | 173.0 |
| Mannitol | 86.50 |
| Sodium Starch Glycolate | 9.000 |
| Magnesium Stearate | 1.500 |
| Total | 300.0 |

Coating

| | |
|---|---|
| Opadry 20A120006 Yellow, Opadry | 10.30 |
| 20A18407 White, or Opadry 20A110008 | |
| Green (HPMC/HPC) | |
| Carnauba Wax | 0.012 |

Pharmaceutical Composition (Tablet)

| | |
|---|---|
| Total | 310.3 |

TABLE 15
Example Pharmaceutical Compositions (Tablet) Comprising
(S)-Formula I HCl (Dose Strengths 50, 75, and 100 mg)

| | | | |
|---|---|---|---|
| Dose Strength (mg) | 50 | 75 | 100 |

| | |
|---|---|
| Ingredient | Composition (mg/tablet) |

Core Tablet, Intra-Granular

| | | | |
|---|---|---|---|
| (S)-Formula I HCl | 60.00 | 90.00 | 120.0 |
| Microcrystalline Cellulose | 23.83 | 35.74 | 47.66 |
| Sodium Starch Glycolate | 1.714 | 2.571 | 3.429 |
| Magnesium Stearate | 0.1714 | 0.2571 | 0.3429 |
| Total | 85.71 | 128.6 | 171.4 |

Core Tablet, Extra-Granular

| | | | |
|---|---|---|---|
| Granules (comprising | 85.71 | 128.6 | 171.4 |
| (S)-Formula I HCl) | | | |
| Microcrystalline Cellulose | 137.9 | 108.8 | 90.24 |
| Mannitol | 68.93 | 54.39 | 30.08 |
| Sodium Starch Glycolate | 6.000 | 6.000 | 6.000 |
| Colloidal Silicon Dioxide | n/a | 0.7500 | 0.7500 |
| Magnesium Stearate | 1.500 | 1.500 | 1.500 |
| Total | 300.0 | 300.0 | 300.0 |

Coating

| | | | |
|---|---|---|---|
| Opadry 20A120006 Yellow, | 10.30 | 10.30 | 10.30 |
| Opadry 20A18407 White, or | | | |
| Opadry 20A110008 | | | |
| Green (HPMC/HPC) | | | |
| Carnauba Wax | 0.01200 | 0.01200 | 0.01200 |

Pharmaceutical Composition (Tablet)

| | | | |
|---|---|---|---|
| Total | 310.3 | 310.3 | 310.3 |

### Methods

The Diagnostic and Statistical Manual of Mental Disorders, Fifth Ed., hereinafter, the “DSM-5”), published by the American Psychiatric Association in 2013, and is incorporated herein by reference, provides a standard diagnostic system upon which persons of skill rely for diagnosis of various diseases and disorders.

The present disclosure provides a method of treating or preventing a central nervous disorder. In some embodiments, the central nervous disorder is a social function disorder. The present disclosure provides a method of treating or preventing a social function disorder comprising administering to a subject in need thereof a therapeutically effective amount of compound of Formula I:

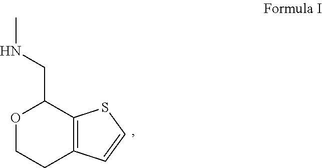

or a pharmaceutically acceptable salt thereof.

The present disclosure provides a method of treating or preventing a social function disorder comprising administering to a subject in need thereof a therapeutically effective amount of compound of Formula I:

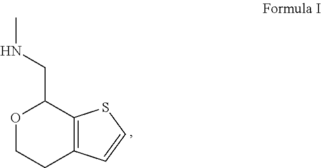

or a pharmaceutically acceptable salt thereof, wherein the compound of Formula I is

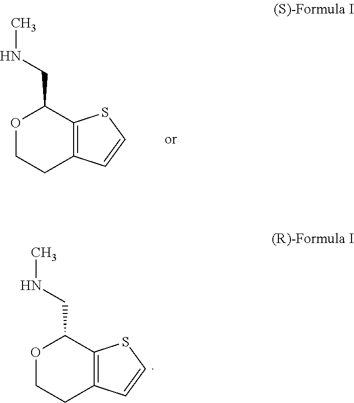

In some embodiments, the social function disorder is a neurodevelopmental disorder, an obsessive-compulsive disorder or a disruptive, impulse-control and conduct disorder. In some embodiments, the social function disorder is a neurodevelopmental disorder. In some embodiments, the social function disorder is an obsessive-compulsive disorder. In some embodiments, the social function disorder is a disruptive, impulse-control and conduct disorder.

In some embodiments, the social function disorder is a language disorder, a speech sound disorder, a childhood-onset fluency disorder (stuttering), a social communication disorder, a developmental coordination disorder, a stereotypical movement disorder, a tic disorder, Tourette's disorder, a persistent (chronic) motor or vocal tic disorder, a provisional tic disorder, another specified tic disorder, an unspecified tic disorder, an obsessive-compulsive disorder, or an impulse-control disorder.

In some embodiments, the social function disorder is a neurodevelopmental disorder. In some embodiments, the neurodevelopmental disorder is a language disorder, a speech sound disorder, a childhood-onset fluency disorder (stuttering), or a social communication disorder.

In some embodiments, the social function disorder is an obsessive-compulsive disorder.

In some embodiments, the social function disorder is a disruptive, impulse-control and conduct disorder. In some embodiments, the disruptive, impulse-control and conduct disorder is an impulse-control disorder.

In some embodiments, the social function disorder is a language disorder, childhood-onset fluency disorder (stuttering), social communication disorder, developmental coordination disorder, stereotypical movement disorder, persistent (chronic) motor or vocal tic disorder, provisional tic disorder, other specified tic disorder, or unspecified tic disorder. In some embodiments, the social function disorder is childhood-onset fluency disorder (stuttering).

In some embodiments, provided is a method of treating or preventing a neurodevelopmental disorder, an obsessive-compulsive disorder or a disruptive, impulse-control and conduct disorder comprising administering to a subject in need thereof a therapeutically effective amount of (S)-Formula I:

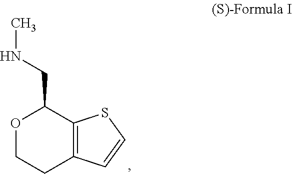

or a pharmaceutically acceptable salt thereof.

In some embodiments, provided is a method of treating a neurodevelopmental disorder, an obsessive-compulsive disorder or a disruptive, impulse-control and conduct disorder comprising administering to a subject in need thereof a therapeutically effective amount of (S)-Formula I:

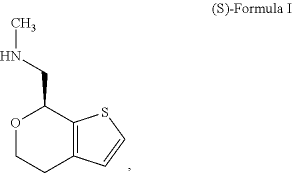

or a pharmaceutically acceptable salt thereof.

In some embodiments, provided is a method of treating a neurodevelopmental disorder, an obsessive-compulsive disorder or a disruptive, impulse-control and conduct disorder comprising administering to a subject in need thereof a therapeutically effective amount of (S)-Formula I:

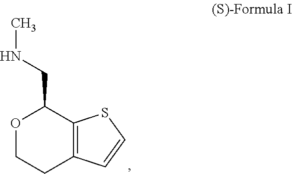

or a pharmaceutically acceptable salt thereof,
wherein the pharmaceutically acceptable salt is a sulfate, pyrosulfate, bisulfate, sulfite, bisulfite, phosphate, monohydrogen-phosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, methylsulfonate, propylsulfonate, besylate, xylenesulfonate, naphthalene-1-sulfonate, naphthalene-2-sulfonate, phenylacetate, phenylpropionate, phenylbutyrate, citrate, lactate, .gamma.-hydroxybutyrate, glycolate, tartrate, or mandelate. In some embodiments, the pharmaceutically acceptable salt is HCl.

In some embodiments, provided is a method of treating a language disorder, a speech sound disorder, a childhood-onset fluency disorder (stuttering), a social communication disorder, a developmental coordination disorder, a stereotypical movement disorder, a tic disorder, Tourette's disorder, a persistent (chronic) motor or vocal tic disorder, a provisional tic disorder, an other specified tic disorder, an unspecified tic disorder, an obsessive-compulsive disorder, or an impulse-control disorder comprising administering to a subject in need thereof a therapeutically effective amount of (S)-Formula I:

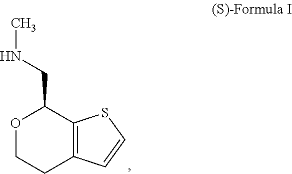

or a pharmaceutically acceptable salt thereof.

In some embodiments, provided is a method of treating a language disorder, a speech sound disorder, a childhood-onset fluency disorder (stuttering), or a social communication disorder comprising administering to a subject in need thereof a therapeutically effective amount of (S)-Formula I:

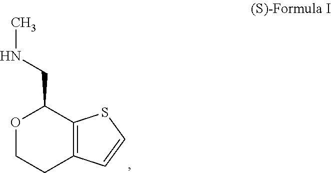

or a pharmaceutically acceptable salt thereof.

In some embodiments, provided is a method of treating a childhood-onset fluency disorder (stuttering) disorder comprising administering to a subject in need thereof a therapeutically effective amount of (S)-Formula I:

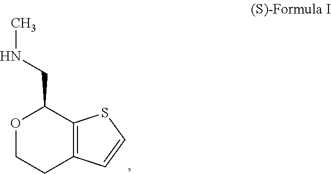

or a pharmaceutically acceptable salt thereof.

In some embodiments, provided is a method of treating an obsessive-compulsive disorder comprising administering to a subject in need thereof a therapeutically effective amount of (S)-Formula I:

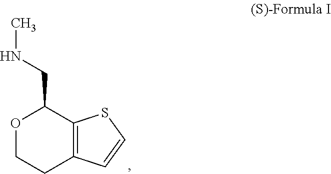

or a pharmaceutically acceptable salt thereof.

In some embodiments, provided is a method of treating or preventing a neurodevelopmental disorder, an obsessive-compulsive disorder or a disruptive, impulse-control and conduct disorder comprising administering to a subject in need thereof a therapeutically effective amount of (S)-Formula I HCl:

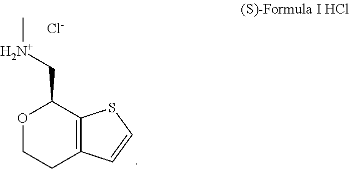

In some embodiments, provided is a method of treating a neurodevelopmental disorder, an obsessive-compulsive disorder or a disruptive, impulse-control and conduct disorder comprising administering to a subject in need thereof a therapeutically effective amount of (S)-Formula I HCl:

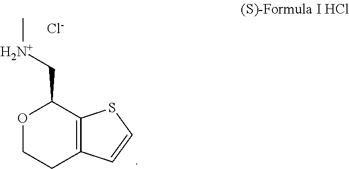

In some embodiments, provided is a method of treating a language disorder, a speech sound disorder, a childhood-onset fluency disorder (stuttering), a social communication disorder, a developmental coordination disorder, a stereotypical movement disorder, a tic disorder, Tourette's disorder, a persistent (chronic) motor or vocal tic disorder, a provisional tic disorder, an other specified tic disorder, an unspecified tic disorder, an obsessive-compulsive disorder, or an impulse-control disorder comprising administering to a subject in need thereof a therapeutically effective amount of (S)-Formula I HCl:

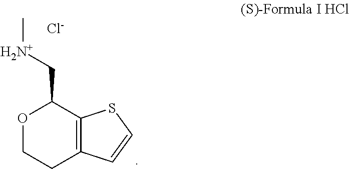

In some embodiments, provided is a method of treating a language disorder, a speech sound disorder, a childhood-onset fluency disorder (stuttering), or a social communication disorder comprising administering to a subject in need thereof a therapeutically effective amount of (S)-Formula I HCl:

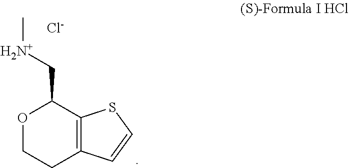

In some embodiments, provided is a method of treating a childhood-onset fluency disorder (stuttering) disorder comprising administering to a subject in need thereof a therapeutically effective amount of (S)-Formula I HCl:

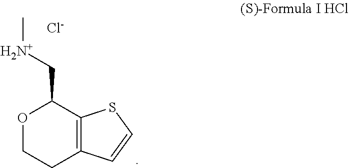

In some embodiments, provided is a method of treating an obsessive-compulsive disorder comprising administering to a subject in need thereof a therapeutically effective amount of (S)-Formula I HCl:

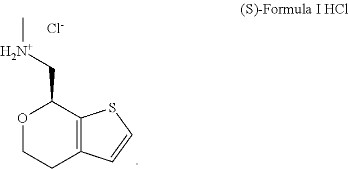

In some embodiments, provided is a method of treating or preventing a neurodevelopmental disorder, an obsessive-compulsive disorder or a disruptive, impulse-control and conduct disorder comprising administering to a subject in need thereof a therapeutically effective amount of crystalline (S)-Formula I HCl Form A:

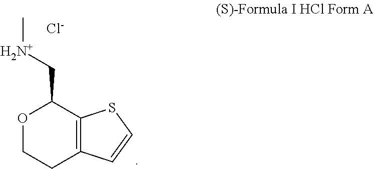

In some embodiments, provided is a method of treating a neurodevelopmental disorder, an obsessive-compulsive disorder or a disruptive, impulse-control and conduct disorder comprising administering to a subject in need thereof a therapeutically effective amount of crystalline (S)-Formula I HCl Form A:

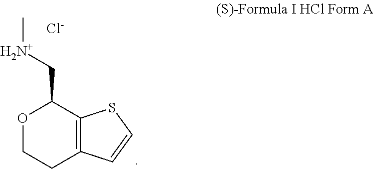

In some embodiments, provided is a method of treating a language disorder, a speech sound disorder, a childhood-onset fluency disorder (stuttering), a social communication disorder, a developmental coordination disorder, a stereotypical movement disorder, a tic disorder, Tourette's disorder, a persistent (chronic) motor or vocal tic disorder, a provisional tic disorder, another specified tic disorder, an unspecified tic disorder, an obsessive-compulsive disorder, or an impulse-control disorder comprising administering to a subject in need thereof a therapeutically effective amount of crystalline (S)-Formula I HCl Form A:

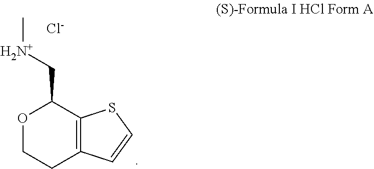

In some embodiments, provided is a method of treating a language disorder, a speech sound disorder, a childhood-onset fluency disorder (stuttering), or a social communication disorder comprising administering to a subject in need thereof a therapeutically effective amount of crystalline (S)-Formula I HCl Form A:

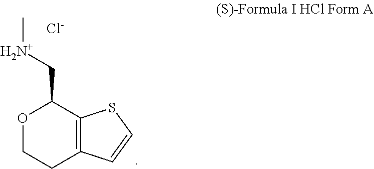

In some embodiments, provided is a method of treating a childhood-onset fluency disorder (stuttering) disorder comprising administering to a subject in need thereof a therapeutically effective amount of crystalline (S)-Formula I HCl Form A:

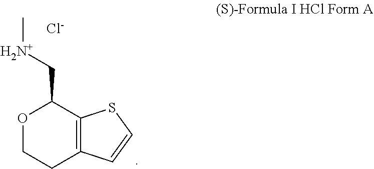

In some embodiments, provided is a method of treating an obsessive-compulsive disorder comprising administering to a subject in need thereof a therapeutically effective amount of crystalline (S)-Formula I HCl Form A:

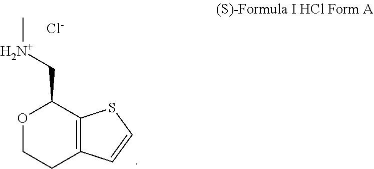

In some embodiments, provided is a method of treating or preventing a neurodevelopmental disorder, an obsessive-compulsive disorder or a disruptive, impulse-control and conduct disorder comprising administering to a subject in need thereof a pharmaceutical composition disclosed herein.

In some embodiments, provided is a method of treating or preventing a neurodevelopmental disorder, an obsessive-compulsive disorder or a disruptive, impulse-control and conduct disorder comprising administering to a subject in need thereof a pharmaceutical composition comprising:
(a) 30 to 125 mg of (S)-Formula I HCl;
(b) 100 to 250 mg of Microcrystalline Cellulose;
(c) 25 to 100 mg of Mannitol;
(d) 5 to 10 mg of Sodium Starch Glycolate; and
(e) 0.75 to 2 mg of Magnesium Stearate.

In some embodiments, provided is a method of treating or preventing a neurodevelopmental disorder, an obsessive-compulsive disorder or a disruptive, impulse-control and conduct disorder comprising administering to a subject in need thereof a pharmaceutical composition comprising:
(a) 30 to 125 mg of (S)-Formula I HCl Form A;
(b) 100 to 250 mg of Microcrystalline Cellulose;
(c) 25 to 100 mg of Mannitol;
(d) 5 to 10 mg of Sodium Starch Glycolate; and
(e) 0.75 to 2 mg of Magnesium Stearate.

In some embodiments, provided is a method of treating or preventing a neurodevelopmental disorder, an obsessive-compulsive disorder or a disruptive, impulse-control and conduct disorder comprising administering to a subject in need thereof a pharmaceutical composition comprising:

| | |
|---|---|
| Ingredient | Composition (mg/tablet) |

Core Tablet

| | |
|---|---|
| (S)-Formula I HCl | 30.00 |
| Microcrystalline Cellulose | 173.0 |
| Mannitol | 86.50 |
| Sodium Starch Glycolate | 9.000 |
| Magnesium Stearate | 1.500 |
| Total | 300.0 |

Coating

| | |
|---|---|
| Opadry 20A120006 Yellow, Opadry | 10.30 |
| 20A18407 White, or Opadry 20A110008 | |
| Green (HPMC/HPC) | |
| Carnauba Wax | 0.012 |

Pharmaceutical Composition (Tablet)

| | |
|---|---|
| Total | 310.3 |

In some embodiments, provided is a method of treating or preventing a neurodevelopmental disorder, an obsessive-compulsive disorder or a disruptive, impulse-control and conduct disorder comprising administering to a subject in need thereof a pharmaceutical composition comprising:

| | |
|---|---|
| Ingredient | Composition (mg/tablet) |

Core Tablet, Intra-Granular

| | |
|---|---|
| (S)-Formula I HCl | 60.00 |
| Microcrystalline Cellulose | 23.83 |
| Sodium Starch Glycolate | 1.714 |
| Magnesium Stearate | 0.1714 |
| Total | 85.71 |

Core Tablet, Extra-Granular

| | |
|---|---|
| Granules (comprising (S)-Formula I HCl) | 85.71 |
| Microcrystalline Cellulose | 137.9 |
| Mannitol | 68.93 |
| Sodium Starch Glycolate | 6.000 |
| Colloidal Silicon Dioxide | n/a |
| Magnesium Stearate | 1.500 |
| Total | 300.0 |

Coating

| | |
|---|---|
| Opadry 20A120006 Yellow, Opadry | 10.30 |
| 20A18407 White, or Opadry 20A110008 | |
| Green (HPMC/HPC) | |
| Camauba Wax | 0.01200 |

Pharmaceutical Composition (Tablet)

| | |
|---|---|
| Total | 310.3 |

In some embodiments, provided is a method of treating or preventing a neurodevelopmental disorder, an obsessive-compulsive disorder or a disruptive, impulse-control and conduct disorder comprising administering to a subject in need thereof a pharmaceutical composition comprising:

| | |
|---|---|
| Ingredient | Composition (mg/tablet) |

Core Tablet, Intra-Granular

| | |
|---|---|
| (S)-Formula I HCl | 90.00 |
| Microcrystalline Cellulose | 35.74 |
| Sodium Starch Glycolate | 2.571 |
| Magnesium Stearate | 0.2571 |
| Total | 128.6 |

Core Tablet, Extra-Granular

| | |
|---|---|
| Granules (comprising (S)-Formula I HCl) | 128.6 |
| Microcrystalline Cellulose | 108.8 |
| Mannitol | 54.39 |
| Sodium Starch Glycolate | 6.000 |
| Colloidal Silicon Dioxide | 0.7500 |
| Magnesium Stearate | 1.500 |
| Total | 300.0 |

Coating

| | |
|---|---|
| Opadry 20A120006 Yellow, Opadry | 10.30 |
| 20A18407 White, or Opadry 20A110008 | |
| Green (HPMC/HPC) | |
| Carnauba Wax | 0.01200 |

Pharmaceutical Composition (Tablet)

| | |
|---|---|
| Total | 310.3 |

In some embodiments, provided is a method of treating or preventing a neurodevelopmental disorder, an obsessive-compulsive disorder or a disruptive, impulse-control and conduct disorder comprising administering to a subject in need thereof a pharmaceutical composition comprising:

| | |
|---|---|
| Ingredient | Composition (mg/tablet) |

Core Tablet, Intra-Granular

| | |
|---|---|
| (S)-Formula I HCl | 120.0 |
| Microcrystalline Cellulose | 47.66 |
| Sodium Starch Glycolate | 3.429 |
| Magnesium Stearate | 0.3429 |
| Total | 171.4 |

Core Tablet, Extra-Granular

| | |
|---|---|
| Granules (comprising (S)-Formula I HCl) | 171.4 |
| Microcrystalline Cellulose | 90.24 |
| Mannitol | 30.08 |
| Sodium Starch Glycolate | 6.000 |
| Colloidal Silicon Dioxide | 0.7500 |
| Magnesium Stearate | 1.500 |
| Total | 300.0 |

Coating

| | |
|---|---|
| Opadry 20A120006 Yellow, Opadry | 10.30 |
| 20A18407 White, or Opadry 20A110008 | |
| Green (HPMC/HPC) | |
| Carnauba Wax | 0.01200 |

Pharmaceutical Composition (Tablet)

| | |
|---|---|
| Total | 310.3 |

In some embodiments, provided is a method of treating an impulse-control disorder comprising administering to a subject in need thereof a therapeutically effective amount of (S)-Formula I:

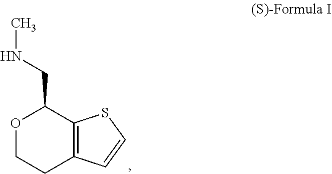

or a pharmaceutically acceptable salt thereof. The present disclosure provides for methods of treating disorders that are responsive to the modulation of D₁ and/or D₂-receptors. While not wishing to be bound by any one theory, the presently disclosed compounds are believed to modulate D₁ and/or D₂ receptors such that the D₁:D₂ ratio in the putamen increases or that the D₂ density is lowered.

In certain embodiments, provided is a method of modulating the density of D₁ and/or D₂-receptors comprising administering a compound of the present disclosure, or a pharmaceutically acceptable salt thereof, to a human.

In certain embodiments, provided is a method of modulating the density of D₁ and/or D₂-receptors to a subject in afflicted with a social function disorder comprising administering a compound of the present disclosure, or a pharmaceutically acceptable salt thereof.

### EXAMPLES

One having ordinary skill in the art would recognize that there is a plurality of ways to test a compound's efficacy in treating a social function disorder. The following non-limiting examples provide study designs to measure efficacy of the compound of Formula I in treating a social function disorder, such as childhood-onset fluency disorder (stuttering). Each study design is incorporated by reference in its entirety.

Study Design 1. Use of the protocol described in clinical trial NCT01684657, entitled, “A Randomized, Placebo-Controlled Study to Evaluate the Efficacy and Tolerability of Asenapine With Flexible Dosing From 5 mg to 20 mg in Adults With Developmental Stuttering,” sponsored by the University of California, Irvine in 2012.

Pharmaceutical compositions comprising between 2.5 to 10 mg of asenapine, or a comparable placebo, were administered daily to 32 subjects, generally not exceeding 5 to 20 mg per day. Outcome of efficacy was objectively measured using a Stuttering Severity Instrument (SSI), where two five-minute speaking samples (conversation and reading from a passage) were captured and evaluated. Additionally, investigators use the Clinical Global Impression scale to evaluate whether or not subjects improved, remained the same or worsened throughout the study.

Subjects were included for satisfying DSM-IV criteria for stuttering, the nature of the stuttering had to have been developmental in origin with the onset prior to ten years of age, and subjects must have had a score of moderate or higher on the Stuttering Severity Instrument-4 (SSI-IV; or SSI-4).

Study Design 2. Use of the protocol described in clinical trial NCT00830154, entitled, “A Study to Assess the Efficacy and Safety of Pagoclone for Adults With Stuttering,” sponsored by Endo Pharmaceuticals in 2009.

Pharmaceutical compositions comprising 0.30 mg or 0.60 mg of pagoclone, or a comparable placebo, were administered twice daily to 321 subjects and evaluated at 8 weeks, 16 weeks, and 24 weeks. Outcome of efficacy was measured, including using the Clinical Global Impressions-Improvement (CGI-I) scale, Patient Global Assessment of Stuttering (PGS-S) assessment, and Liebowitz Social Anxiety Scale (LSAS) scores.

Subjects were included for presenting with a history of stuttering with onset prior to age eight years old and the stuttering severity must have been notable for more than 3 syllables stuttered on a reading and conversation task at screening and with at least 2% contributed individually from conversational and reading tasks.

Study Design 3. Use of the protocol described in clinical trial NCT00239915 entitled, “Safety and Efficacy Study of the Investigational Drug Pagoclone, in the Treatment of Persistent Developmental Stuttering (PDS),” sponsored by Pharmacology Research Institute in 2005.

Pharmaceutical compositions comprising pagoclone, were administered to subjects and evaluated at 8 weeks, followed by a 52 week open label extension.

Subjects were included for presenting with Persistent Developmental Stuttering (PDS) with criteria set forth in the DSM-IV-TR; symptoms starting before age eight; a total score of 18-36 on the Stuttering Severity Instrument-3 (SSI-3); and English speaking with at least an 8^{th} grade education; able to understand and cooperate with study requirements with assistance.

Study Design 4. Use of the protocol described in clinical trial NCT00216255 entitled, “EXPRESS: Examining Pagoclone for Persistent Developmental Stuttering Study,” sponsored by Endo Pharmaceuticals in 2005.

A flexible dosing titration regimen from 0.15 mg pagoclone administered twice daily (BID), titrated at two-weeks to 0.30 mg pagoclone administered twice daily for an additional six-weeks, or a comparable placebo, was administered to 120 subjects to study Persistent Developmental Stuttering (PDS) in patients 18 to 65 years of age over an eight-week period, followed by a 53 week open label treatment extension period. Outcome of efficacy was objectively measured using the Stuttering Severity Instrument-3 (SSI-3) Frequency and Duration Subscore, the Subjective Screening of Stuttering (SSS) Severity Subscore, and the treatment and week 8 visits.

Subjects were included for presenting with Persistent Developmental Stuttering (PDS) with criteria set forth in the DSM-IV-TR; symptoms starting before age eight; a total score of 18-36 on the Stuttering Severity Instrument-3 (SSI-3); and English speaking with at least an 8^{th} grade education; able to understand and cooperate with study requirements with assistance.

Study Design 5. Use of the protocol described in clinical trial NCT02909088 entitled, “Efficacy and Tolerability of Ecopipam in Adults With Childhood Onset Fluency Disorder (Stuttering),” sponsored by Gerald Maguire, MD in 2016

Pharmaceutical compositions comprising 50 to 100 mg of ecopipam were administered to 10 subjects. Initially, subjects started at 50 mg of ecopipam and if no improvement was found after 14 days, the dose was increased to 100 mg of ecopipam. Outcome of efficacy was measured using Stuttering Severity Instrument-4 (SSI-IV); Clinical Global Impression Scale-Severity (CGI-S); Subjective Stuttering Scale (SSS); Overall Assessment of the Speaker's Experience of Stuttering (OASES); Montgomery Asberg Depression Rating Scale (MADRS); Barnes Akathisia Scale (BAS); Abnormal Involuntary Movement Scale (AIMS): Columbia-Suicide Severity Rating Scale (C-SSRS); and Simpson Angus Scale (SAS).

Subjects were included for presenting with childhood onset fluency disorder (stuttering) with criteria set forth in the DSM-IV; symptoms starting before age ten; score of moderate or higher on the Stuttering Severity Instrument-4 (SSI-4); and have a MADRS score of ≤13 (normal mood).

Study Design 6. Use of a protocol described in “Procedures Used for Assessment of Stuttering Frequency and Stuttering Duration” as published in Clinical Linguistics & Phonetics, Volume 27, Issue 12, pages 853-861, 2013 and written by Jani, L et al.

Jani, L. et al describe methods of assessing stuttering, including instruments for real-time judgments. Methods for assessing stuttering include: syllable-based (e.g., Stuttering Severity Instrument-3 (SSI-3); Stuttering Severity Instrument-4 (SSI-4); disfluency-based analyses (e.g., frequency and durational measures of stuttering, transcript-based and live procedures using Systematic Disfluency Analysis (SDA), TrueTalk used by Lidcombe, Computerized Scoring of Stuttering Severity version 2 (CSSS-2.0), Stuttering Measurement System (SMS), and phone applications (e.g., Smarty Ears—The Disfluency Index Counter; The Duo Counter).

SSI-3 can be used (1) as part of a diagnostic evaluation, (2) for tracking changes in severity in severity during and after treatment, (3) to describe the severity distribution in experimental groups that include people who stutter, (4) to validate other stuttering measures, (5) to estimate statistical risk of whether an eight-year-old child who stutters will persist or recover by teenage years, and (6) to distinguish groups of children who stutter from their fluent peers.

Additional study designs can be found, for example, in:

Study Design 7. Use of a protocol described in “Influence of Methylphenidate on the Frequency of Stuttering: A Randomized Controlled Trial” as published in the Annals of Pharmacotherapy, Volume 49, Issue 10, pages 1096-1104, 2015.

Study Design 8. Use of a protocol described in “Risperidone for the treatment of stuttering” as published in the Journal of Clinical Psychopharmacology, Volume 20, Issue 4, pages 479-482, 2000.

Study Design 9. Use of a protocol described in “Pharmacological agents for developmental stuttering in children and adolescents: a systematic review” as published in the Journal of Clinical Psychopharmacology, Volume 31, Issue 6, pages 740-744, 2011.

All of the U.S. patents, U.S. patent application publications, U.S. patent applications, foreign patents, foreign patent applications, and non-patent publications referred to in this specification are incorporated herein by reference, in their entirety to the extent not inconsistent with the present description.

1. A method of treating or preventing a social function disorder, wherein the social function disorder is a language disorder, a speech sound disorder, a childhood-onset fluency disorder (stuttering), or a social communication disorder, comprising administering to a subject in need thereof a therapeutically effective amount of 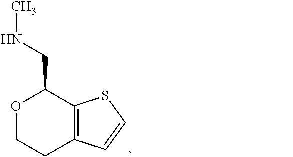
or a pharmaceutically acceptable salt thereof.

2. A method of treating or preventing a social function disorder, wherein the social function disorder is a neurodevelopmental disorder, an obsessive-compulsive disorder or a disruptive, impulse-control and conduct disorder comprising administering to a subject in need thereof a therapeutically effective amount of 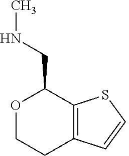
or a pharmaceutically acceptable salt thereof.

3. A method of treating or preventing a social function disorder, wherein the social function disorder is a a developmental coordination disorder, a stereotypical movement disorder, a tic disorder, Tourette's disorder, a persistent (chronic) motor or vocal tic disorder, a provisional tic disorder, other specified tic disorder or unspecified tic disorder, comprising administering to a subject in need thereof a therapeutically effective amount of 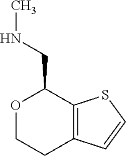
or a pharmaceutically acceptable salt thereof.

4. (canceled)

5. (canceled)

6. (canceled)

7. A method of treating or preventing a social function disorder according to claim 1, comprising administering to a subject in need thereof a pharmaceutical composition comprising:
(a) 25 mg to 125 mg of a compound of the following formula: 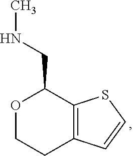
or a pharmaceutically acceptable salt thereof, and optionally additionally comprising:
(b) one or more filler;
(c) one or more disintegrant; and
(d) one or more lubricant.

8. The method of claim 7 wherein the one or more filler is any one or more of microcrystalline cellulose, mannitol, and xylitol.

9. (canceled)

10. (canceled)

11. The method according to claim 7 comprising administering to a subject in need thereof a pharmaceutical composition comprising:
(a) 30 to 125 mg of 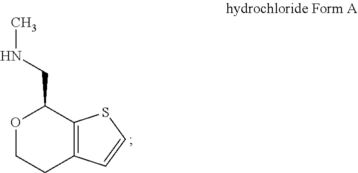
(b) 100 to 250 mg of Microcrystalline Cellulose;
(c) 25 to 100 mg of Mannitol;
(d) 5 to 10 mg of Sodium Starch Glycolate; and
(e) 0.75 to 2 mg of Magnesium Stearate.
